(19) United States
(12) Patent Application Publication
(10) Pub. No.: US 2024/0041838 A1
(43) Pub. Date: Feb. 8, 2024

(54) METHODS AND COMPOSITIONS FOR INHIBITION OF NEUTROPHIL RECRUITMENT IN MYCOCARDIAL ISCHEMIA-REPERFUSION INJURY

(71) Applicant: University of Connecticut, Farmington, CT (US)

(72) Inventors: Zhichao Fan, Farmington, CT (US); Bruce T. Liang, Avon, CT (US)

(21) Appl. No.: 18/362,485

(22) Filed: Jul. 31, 2023

Related U.S. Application Data
(60) Provisional application No. 63/393,284, filed on Jul. 29, 2022.

(51) Int. Cl. A61K 31/4196; A61P 9/10

(52) U.S. Cl. CPC A61K 31/4196; A61P 9/10

(57) ABSTRACT

Compositions and methods for the treatment of a human subject in need of treatment for a neutrophil-related condition, in particular who has had a stroke or myocardial ischemia reperfusion injury, by administering to the subject a pharmaceutical composition including a compound of Formula 1,

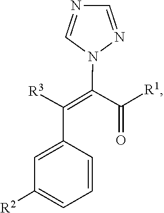

wherein the substituents are as described herein, and in particular wherein the compound is 4,4-dimethyl-1-(3-nitrophenyl)-2-(1H-1,2,4-triazol-1-yl)-1-penten-3-one.

### CROSS-REFERENCE TO RELATED APPLICATION

This application claims priority under 35 U.S.C. § 119(e) to U.S. Provisional Patent Application Ser. No. 63/393,284, filed on Jul. 29, 2022, the contents of which are hereby incorporated by reference in their entirety.

### STATEMENT REGARDING FEDERALLY SPONSORED RESEARCH & DEVELOPMENT

This invention was made with government support under HL152710, HL145454, and HL156322 awarded by the National Institutes of Health. The government has certain rights in the invention.

### BACKGROUND

### 1. Field of the Disclosure

The subject disclosure relates to methods and compositions for prevention and/or treatment of neutrophil-related conditions, including myocardial ischemia-reperfusion injury.

### 2. Description of the Related Art

Neutrophils are the most abundant leukocytes in humans and serve as the first responders to inflammation and infection. An intrinsic neutrophil defect leads to pathologies, such as leukocyte adhesion deficiency syndromes. Abnormal activation of neutrophils is critically involved in most inflammatory diseases, including auto-immune diseases. Neutrophils are further implicated ischemia-reperfusion (I/R) injury associated with treatment of myocardial infarction (MI, i.e., heart attack). MI is the irreversible death (necrosis and apoptosis) of heart muscle secondary to prolonged lack of oxygen supply (ischemia). Approximately 1.5 million cases of MI occur annually in the United States. Examples of myocardial infarction include ST elevation myocardial infarction (STEMI), non-ST elevation myocardial infarction (NSTEMI) and acute myocardial infarction. Reperfusion following ischemia results in an influx of circulating immune cells, such as neutrophils and monocytes, to the injured myocardium. While ischemia caused by occlusion of the coronary artery leads to infarcted myocardium, reopening of the blocked artery may further contribute significantly to cardiac injury known as myocardial ischemia reperfusion injury. Clinically in patients, such reperfusion injury occurs after opening of the blocked coronary artery via percutaneous coronary intervention with a stent or thrombolytic medication. Thus, preventing myocardial ischemia reperfusion injury may reduce infarct size or prevent deterioration of cardiac function.

Thus, improved pharmacological therapy for neutrophil-related conditions such as stroke, particularly ischemic stroke, and myocardial ischemia reperfusion injury represent areas of unmet need in the art.

### SUMMARY

In an aspect, disclosed is a method for treating or preventing a neutrophil-related acute inflammatory condition in a subject in need thereof, the method comprising: providing a pharmaceutical composition comprising a compound of Formula 1, and/or a pharmaceutically acceptable salt and/or formulation thereof

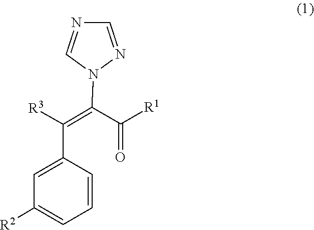

wherein, in Formula 1, R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl, R² is hydrogen, cyano, halo, or nitro, and R³ is hydrogen, cyano, halo, nitro, C₁-C₄ alkyl, C₁-C₄ haloalkyl, C₃-C₆ cycloalkyl, C₂-C₇ heterocycloalkyl, C₆-C₁₂ aryl, or C₂-C₁₁ heteroaryl; and administering the pharmaceutical composition to the subject.

In another aspect the method is for treating or preventing myocardial ischemia-reperfusion injury in a subject in need thereof, the method including: providing a pharmaceutical composition including a compound of Formula 1, and/or a pharmaceutically acceptable salt and/or formulation thereof, preferably a composition including Nexinhib 20 and/or a pharmaceutically acceptable salt and/or formulation thereof; and administering the composition to the subject.

In another aspect, disclosed is a composition for treating or preventing myocardial ischemia-reperfusion injury in a subject including a compound of Formula 1, and/or a pharmaceutically acceptable salt and/or formulation thereof, preferably Nexinhib 20 and/or a pharmaceutically acceptable salt and/or formulation thereof.

These and other aspects of the present invention are described in more detail below in the Figures, Detailed Description, Examples, Definitions, and Claims.

### BRIEF DESCRIPTION OF THE DRAWINGS

Reference will now be made to the accompanying drawings, which are not necessarily drawn to scale, and wherein:

FIGS. 1A-1D show that Nexinhib20 inhibits neutrophil adhesion in response to IL-8. Purified human neutrophils were rolled on the substrate of P-selectin and ICAM-1 with or without IL-8 under a shear stress of 6 dyn·cm⁻². FIG. 1A shows the tracks of rolling neutrophils (n≥29) treated with Nexinhib20 (10 M, RT 1 hour) or vehicle control (CT). FIG. 1B shows cumulative frequency and FIG. 1C shows neutrophil rolling velocity (Mean±SD; n=30). FIG. 1D shows the number of arrested neutrophils (Mean±SD) in 9 fields-of-view. n.s. (non-significant) p>0.05, ****p<0.0001 by 2-way ANOVA followed by Tukey's multiple comparisons test.

FIGS. 2A-2I show that Nexinhib20 inhibits β2 integrin activation and intracellular Ca²⁺ signal after stimulation by IL-8. FIG. 2A and FIG. 2B show a homogenous binding assay: typical graphs showing the dynamic expression (the moving average of median fluorescence intensity, MFI) of mAb24 (FIG. 2A, high-affinity β2 integrins) and KIM127 (FIG. 2B, extended β2 integrins) epitopes on purified human neutrophils pretreated with Nexinhib20 (10 μM, RT 1 hour, the cyan curve in FIG. 2A or magenta curve in FIG. 2B) or vehicle control (DMSO, the blue curve in FIG. 2A or purple curve in FIG. 2B). Fluorescent-labeled antibody (mAb24-AF488 in FIG. 2A or KIM127-DL550 in FIG. 2B) was added 10 seconds after initiation to stain neutrophils. IL-8 was added 5 minutes after initiation to induce integrin activation (high-affinity and extension). The background, in which neutrophils were not stimulated with IL-8, was shown as control (CT) in gray curves. FIGS. 2C-2E are the bar graphs showing the MFI of mAb24 (FIG. 2C), KIM127 (FIG. 2D), or TS1/18 (FIG. 2E, total β2 integrins) on neutrophils treated with vehicle (DMSO) or Nexinhib20 10 min after IL-8 stimulation (IL-8) or vehicle (PBS, CT). MF of isotype control staining was subtracted as the background. Mean±SD, n=6 replicates. FIG. 2F and FIG. 2G show a percentage of high-affinity (FIG. 2F, mAb24) and extended (FIG. 2G, KIM127) β2 integrins on neutrophils treated with vehicle (DMSO) or Nexinhib20 10 min after IL-8 stimulation (IL-8) or vehicle (PBS, CT). Since mAb24, KIM127, and TS1/18 are all IgG1 isotypes, and we used the same secondary antibody, the percentage of high-affinity and extended β2 integrins can be calculated by dividing the MFI of mAb24 and KIM127 by the MFI of TS1/18. Mean±SD, n=6 replicates. FIG. 2H is a typical graph showing the dynamics (the moving average of Fluo-4 MFI) of intracellular Ca²⁺ in neutrophils treated with Nexinhib20 (10 μM, RT 1 hour, the cyan curve) or vehicle control (DMSO, the red curve) stimulated by IL-8 (added at minute 1) or not (the gray curve). FIG. 2I shows an intracellular calcium of neutrophils by Fluo-4 MR (Mean±SD; n=3 individual experiments) without (CT) or with IL-8 simulation and without or with Nexinhib20. ns (non-significant) p>0.05, *p<0.05, **p<0.01, ***p<0.001, ****p<0.0001 by unpaired student's t-test.

FIGS. 3A-3H show that Nexinhib20 inhibits Rac-1 activation by antagonizing GTP binding. FIG. 3A and FIG. 3B show a representative western blot image (FIG. 3A) and quantifications (FIG. 3B) of active PAK-PBD pulled-down Rac-1-GTP (Rac-1 PD) and total Rac-1 (Rac-1 input) in Nexinhib20 incubated (+) (10 μM, RT 1 hour) or control (−) HL60-CXCR2 cells stimulated with (+) or without (−) IL-8 (1 μg·mL⁻¹, 1 minute, RT). FIG. 3C and FIG. 3D show a representative western blot image (FIG. 3C) and quantifications (FIG. 3D) of active PAK-PBD pulled-down Rac-1-GTP (Rac-1 PD) and total Rac-1 (Rac-1 input) in Nexinhib20-incubated (+) (10 μM, RT 1 hour) or control (−) HL60 cells stimulated with (+) or without (−) fMLP (100 nM, 1 minute, RT). FIG. 3E and FIG. 3F show a representative western blot image (FIG. 3E) and quantifications (FIG. 3F) of active PAK-PBD pulled-down Rac-1-GTP (Rac-1 PD) and total Rac-1 (Rac-1 input) in Nexinhib20-incubated (+) (10 μM, RT 1 hour) or control (−) human neutrophils stimulated with (+) or without (−) IL-8 (1 μg·mL⁻¹, 1 minute, RT). Mean±SD from 5 independent experiments in FIG. 3B and FIG. 3D, and 3 independent experiments in FIG. 3F. ns (non-significant) p>0.05, *p<0.05, **p<0.01 by one-way ANOVA followed by Tukey's multiple comparisons test. FIG. 3G is a representative western blot image showing the amount of PAK-PBD pulled-down Rac-1-GTP (Rac-1 PD) when purified His-tag Rac-1 was incubated with GTPγS (non-hydrolysable GTP analog) in vitro in the presence of different concentrations of Nexinhib20 (shown in μM) or not (the same amount of DMSO vehicle added). His-tag Rac-1 incubated with GDP was used as a negative control. FIG. 3H is the fitting curve (Absolute IC50, X is concentration in Prism) showing the inhibition efficiency of Nexinhib20 on the Rac-GTP interaction. Individual values from 3 independent experiments are shown. The values were normalized by setting GDP-added samples to 0 and GTPγS-added vehicle samples to 100. Zoomed-in graph (right) showing that the IC50 was around 29.3 μM.

FIGS. 4A-4G show that Nexinhib20 limits neutrophil recruitment in the heart during mouse myocardial I/R injury. FIG. 4A is a representative multi-photon microscopy images showing the recruitment of EGFP-labeled leukocytes (most of them are neutrophils) at the coronary artery (CD31-AF594 labeled) of LysM-EGFP myocardial I/R (35/60 minutes) mice without (left) or with (right) Nexinhib20 (1 μmol per mouse) administration. In these images, peripheral blood in the heart was washed out by infusing PBS through the aorta; Thus, LysM-EGFP⁺ cells visualized in the images were adhered to the vessel wall or infiltrated into the tissue. FIG. 4B shows a mean±SD of EGFP MFI in coronary arteries of mice administered with vehicle control and Nexinhib20. n=46 and 28 fields-of-view from 9 vehicle control and 7 Nexinhib20 treated mice, respectively. FIG. 4C is a representative flow cytometry plots showing percentages of Ly6G⁺ neutrophils in LysM-EGFP⁺ leukocytes in I/R heart (left) and blood (right) of mice without (top) or with (bottom) Nexinhib20 (1 μmol per mouse) administration. FIG. 4D and FIG. 4E show a mean±SD of neutrophil percentages in LysM-EGFP⁺ leukocytes in I/R heart (FIG. 4D) and blood (FIG. 4E) of mice treated with Nexinhib20 (1 μmol per mouse) or vehicle. n=6 mice. FIG. 4F shows a mean±SD of neutrophil counts (left) and percentages in CD45⁺ live leukocytes (right) in IR heart of mice treated with Nexinhib20 (1 μmol per mouse) or vehicle. n=6 mice. FIG. 4G shows a mean±SD of neutrophil counts in blood of myocardial I/R mice treated with Nexinhib20 (1 μmol per mouse) or vehicle. n=6 mice. *p<0.05, **p<0.01 by unpaired Student's t-test.

FIGS. 5A-5E show that Nexinhib20 decreases the infarct area in mouse myocardial I/R injury. FIG. 5A is a representative images showing TTC-phthalo-blue-stained heart serial sections from myocardial I/R (35 min/24 hours) mice administered with Nexinhib20 (I/R Nexinhib20, the bottom row) or vehicle control (I/R vehicle, the third row). Sham control (the top row) and myocardial ischemia-reperfusion mice without any administration (I/R control, the second row) are also shown. FIG. 5B shows a mean±SD of the infarct area percentage in the area of risk from n=12 mice per group. FIG. 5C shows a mean±SD of the area of risk percentage from n=12 mice per group. ns (non-significant) p>0.05, *p<0.05 by one-way ANOVA followed by Tukey's multiple comparisons test. FIG. 5D and FIG. 5E show an analysis of left ventricle echocardiogram before and after myocardial I/R (35 min/24 hours). Mean±SD of ejection fraction (FIG. 5D) and fraction shortening (FIG. 5E) from n=11 vehicle-treated mice and n=12 Nexinhib20-treated mice. ns p>0.05, **p<0.01, **** p<0.0001 by two-way ANOVA followed by Tukey's multiple comparisons test.

FIGS. 6A-6C show the effects of Nexinhib20 on cell viability and ROS production. FIG. 6A shows Neutrophil viability upon 1-hour incubation with different concentrations of Nexinhib20. FIG. 6B shows ROS production dynamics of isolated human neutrophils treated with Nexinhib20 or vehicle (DMSO). Neutrophils were seeded on ICAM-1-coated wells of a 96-well plate. ROS production after PMA stimulation or vehicle (PBS) was recorded every 5 min. Mean±SD, n=3 replicates. *p<0.05, **p<0.01, ****p<0.0001 comparing vehicle PMA and Nexinhib20 PMA by 2-way ANOVA followed by Šidák's multiple comparisons test. FIG. 6C shows ROS production dynamics of isolated human neutrophils with or without CD18 blockade. Neutrophils were seeded on ICAM-1-coated wells of a 96-well plate. ROS production after PMA stimulation or vehicle was recorded every 5 min. Mean±SD, n=3 replicates.

FIGS. 7A and 7B show the pharmacokinetics of Nexinhib20 after i.p. injection in mice. FIG. 7A shows plasma Nexinhib20 concentrations sampling at 3 min, 15 min, 45 min, 2 h, 4 h, 8 h, and 24 h. FIG. 7B shows plasma Nexinhib20 concentrations sampling at 3 min, 15 min, 45 min, 2 h, and 4 h. Nexinhib20 concentration values measured from 3 mice were shown individually. A one phase exponential decay fitting curve was shown. The half-life of Nexinhib20 in vivo is from 0.1921 to 0.2720 h (95% CI).

### DETAILED DESCRIPTION

Neutrophils are important for mediating inflammatory responses. Inhibiting neutrophil recruitment is an attractive approach for preventing inflammatory injuries, including myocardial ischemia-reperfusion (I/R) injury, which exacerbates cardiomyocyte death after primary percutaneous coronary intervention in acute myocardial infarction. In particular, β2 integrin activation is critical for neutrophil recruitment. Therefore, regulating β₂ integrin signaling is a potential path to reduce inflammatory injury. Several GTPases are involved in β₂ integrin signaling, such as ras homolog gene family (Rho) GTPases and Ras-related protein 1 (Rap1) GTPases. Several neutrophil exocytosis inhibitors (nexinhibs) were identified by Förster resonance energy transfer (FRET)-based screens that targeted the interaction of the small GTPase Rab27a and its effector JFC1. The small GTPase Rab27a is an essential regulator of neutrophil exocytosis. Molecular docking analysis showed that a compound of the following formula

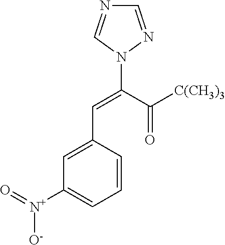 [SMILES: CC(C)(C)C(=O)/C(=C\c1cccc([N+](=O)[O-])c1)n1cncn1]

(4,4-dimethyl-1-(3-nitrophenyl)-2-(1H-1,2,4-triazol-1-yl)-1-penten-3-one, also known as “Nexinhib20”) may interact with an epitope formed by I10, K11, R90, D91, M93, Y122, S123, I181, R184, M185, and S188 of Rab27a. Compounds of this type (“nexinhibs”) did not interact with another small GTPase, Rab11. Whether nexinhibs affect the function of other GTPases, especially those involved in the integrin activation signaling pathway, is unknown. Identifying a nexinhib that inhibits both integrin activation and exocytosis may serve as a dual-functional drug for treating inflammatory diseases.

Myocardial I/R injury exacerbates cardiomyocyte death after primary percutaneous coronary intervention in acute myocardial infarction. Neutrophils are recruited to cardiac tissue during myocardial I/R injury where they worsen injury. They mediate cardiomyocyte death by causing vascular plugging, releasing degradative enzymes, and generating reactive oxygen species (ROS). Neutrophil depletion in mice and dogs with myocardial I/R injury showed significant benefits in reducing infarct size. Inhibiting or deleting myeloperoxidase, which is mainly expressed by neutrophils, improves myocardial function after I/R injury. The neutrophil recruitment cascade includes rolling, slow-rolling, arrest, spreading, intravascular crawling, trans-endothelial migration, and migration to the site of inflammation; and β2 integrins play critical roles in most steps of the neutrophil recruitment cascade. Blocking neutrophil recruitment in mouse knockouts of β2 integrin (CD18) or its ligand, intercellular adhesion molecule 1 (ICAM-1), significantly reduced infarct size after myocardial I/R injury. Similar results were observed in β2 integrin antibody blocking experiments in primate, pig, dog, rabbit, and rat hearts. Thus, targeting β2 integrin activation might be a potential path to reduce myocardial I/R injury.

It has been found by the inventors hereof that a compound of Formula 1, in particular, Nexinhib20, can inhibit human neutrophil adhesion and β2 integrin activation by targeting Ras-related C3 botulinum toxin substrate 1 (Rac-1) GTPase, and that a compound of Formula 1, in particular Nexinhib20 can limit neutrophil recruitment and decrease infarct size after mouse myocardial I/R injury. In particular, it has been found that a neutrophil exocytosis inhibitor Nexinhib20 inhibits not only exocytosis but also neutrophil adhesion by limiting β2 integrin activation. Using a microfluidic chamber, it was found that Nexinhib20 inhibited interleukin 8 (IL-8)-induced β2 integrin-dependent human neutrophil adhesion under flow. Using a dynamic flow cytometry assay, it was further discovered that Nexinhib20 suppresses intracellular calcium flux and β2 integrin activation after IL-8 stimulation. Western blots of Rac-1-GTP pull-down assays confirmed that Nexinhib20 inhibited Rac-1 activation in leukocytes. An in vitro competition assay showed that Nexinhib20 antagonized the binding of Rac-1 and GTP. Using a mouse model of myocardial I/R injury, Nexinhib20 administration after ischemia and before reperfusion significantly decreased neutrophil recruitment and infarct size. These results highlight the translational potential of a compound of Formula 1, in particular, Nexinhib20 as a dual-functional neutrophil inhibitory drug to prevent myocardial I/R injury.

Compounds of Formula 1 are of Formula 1

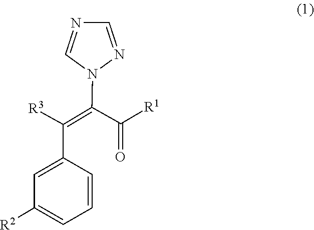

wherein, in Formula 1,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl;
R² is hydrogen, cyano, halo, or nitro; and
R³ is hydrogen, cyano, halo, nitro, C₁-C₄ alkyl, C₁-C₄ haloalkyl, C₃-C₆ cycloalkyl, C₂-C₇ heterocycloalkyl, C₆-C₁₂ aryl, or C₂-C₁₁ heteroaryl.

In an aspect in Formula 1, R¹ is hydrogen, cyano, halo, C₁-C₆ alkyl, C₁-C₆ haloalkyl, C₃-C₆ cycloalkyl, C₂-C₇ heterocycloalkyl, C₆-C₅ aryl, or C₂-C₇ heteroaryl. In another aspect, R¹ is C₁-C₆ alkyl or C₁-C₆ haloalkyl. In another aspect, R¹ is C₁-C₆ alkyl, or C₄ alkyl.

In another aspect in Formula 1. R² is hydrogen, cyano, halo, or nitro. Preferably R² is nitro.

In another aspect in Formula 1, R³ is hydrogen, cyano, halo, C₁-C₄ alkyl, C₁-C₄ haloalkyl, C₃-C₆ cycloalkyl, C₂-C₇ heterocycloalkyl, C₆-C₁₂ aryl, or C₂-C₁₁ heteroaryl. In another aspect, R³ is hydrogen, cyano, halo, C₁-C₃ alkyl, or C₁-C₃ haloalkyl. In still another aspect, R³ is hydrogen, C₁-C₃ alkyl, or C₁-C₃ haloalkyl.

For example, in Formula 1, R¹ is C₁-C₆ alkyl or C₁-C₆ haloalkyl; R² is hydrogen, cyano, halo, or nitro, preferably nitro; and R³ is hydrogen, cyano, halo, C₁-C₄ alkyl, C₁-C₄ haloalkyl, C₃-C₆ cycloalkyl, C₂-C₇ heterocycloalkyl, C₆-C₁₂ aryl, or C₂-C₁₁ heteroaryl. In another aspect, R¹ is C₁-C₆ alkyl; R² is nitro; and R³ is hydrogen, cyano, halo, C₁-C₃ alkyl, or C₁-C₃ haloalkyl.

In a specific example, the compound of Formula 1 is Formula 1a

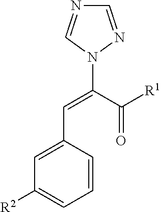

wherein in Formula 1a,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl; and
R² is hydrogen, cyano, halo, or nitro.

For example in Formula 1a, R¹ is C₁-C₆ alkyl or C₁-C₆ haloalkyl; and R² is hydrogen, cyano, halo, or nitro, preferably nitro. In another aspect, R¹ is C₂-C₆ alkyl or C₂-C₆ haloalkyl; and R³ is nitro.

A specific compound of formula 1a is Nexinhib20, in which R¹ is tert-butyl and R² is nitro in Formula 1a.

The compounds of Formula 1 (which as used herein include compounds of Formula 1a) can have one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with double bonds can occur in Z- and E-forms, with all isomeric forms of the compounds being included in the present disclosure. In these situations, the single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

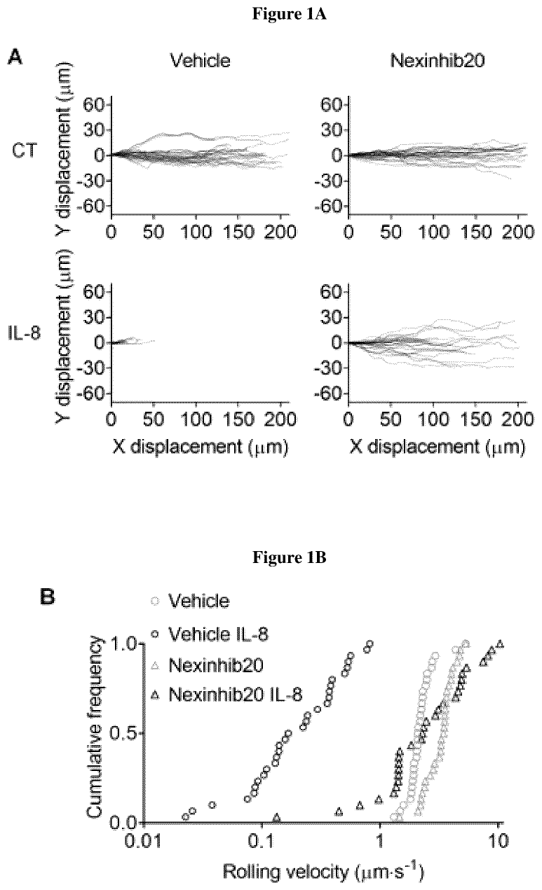

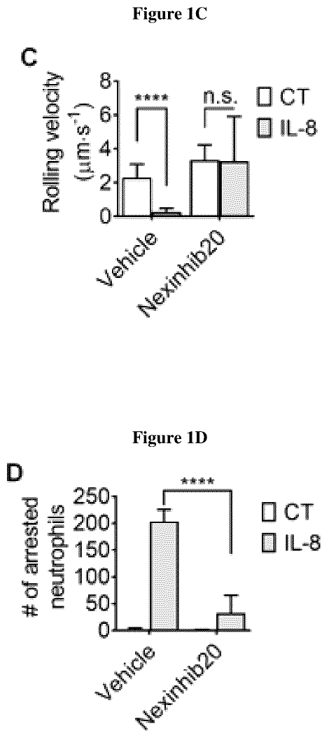

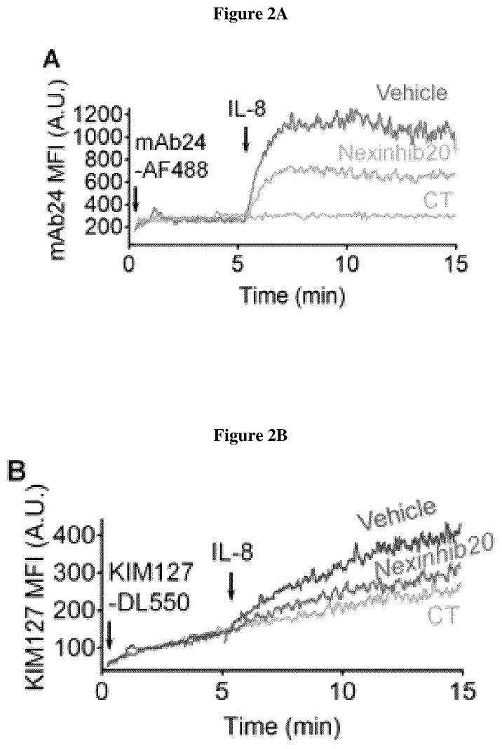

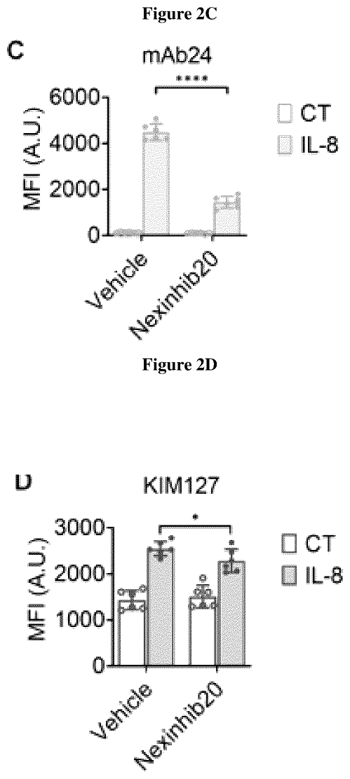

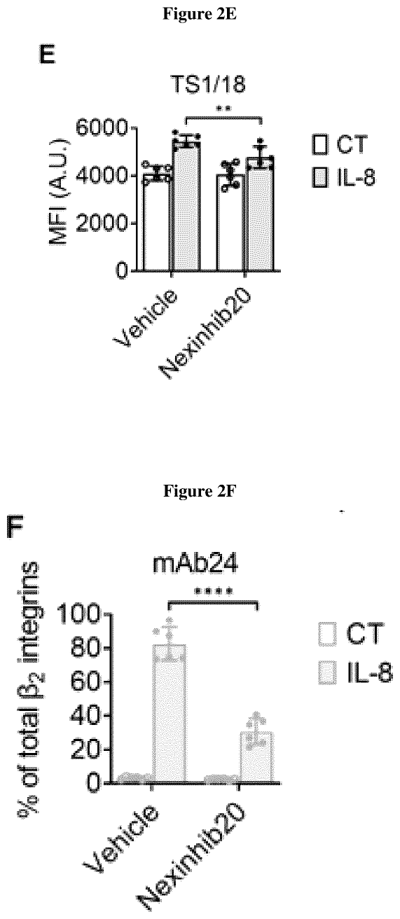

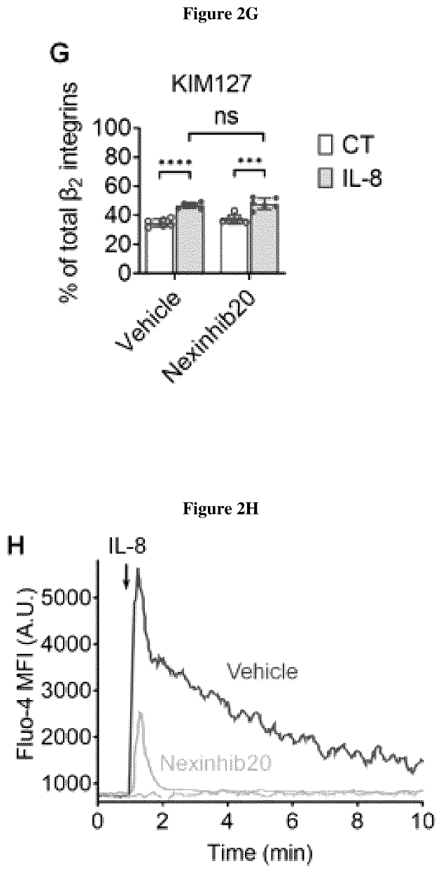

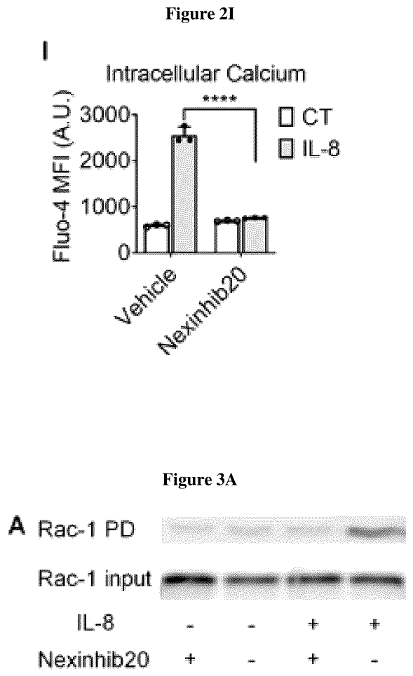

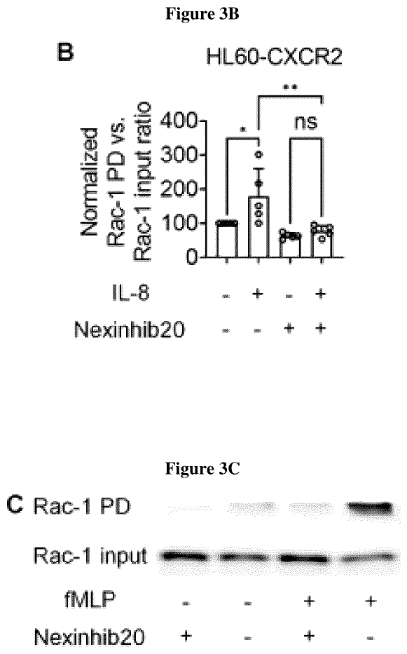

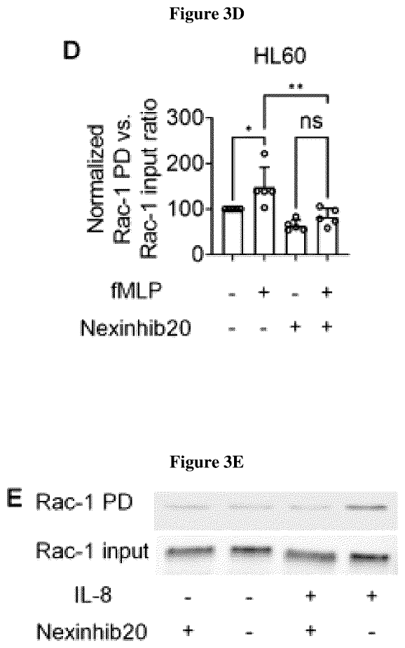

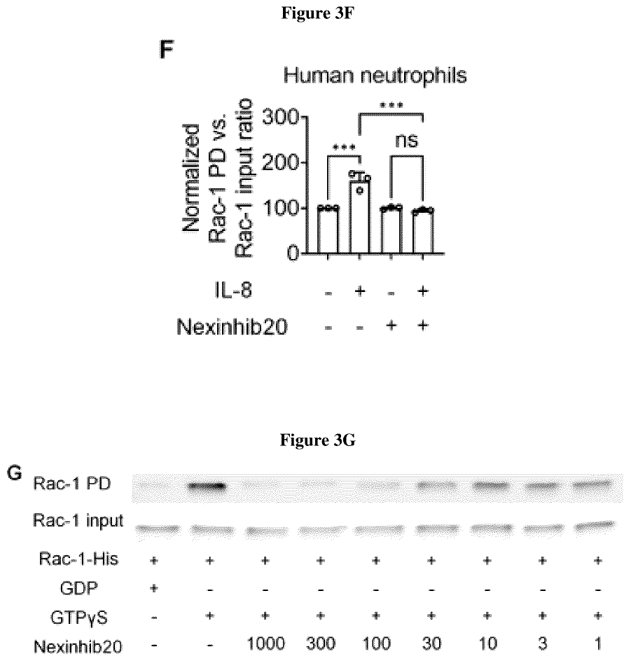

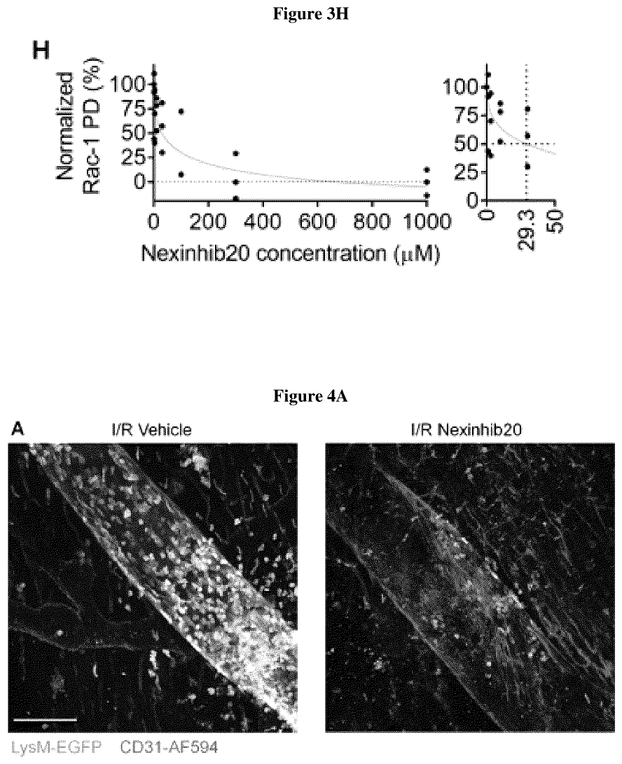

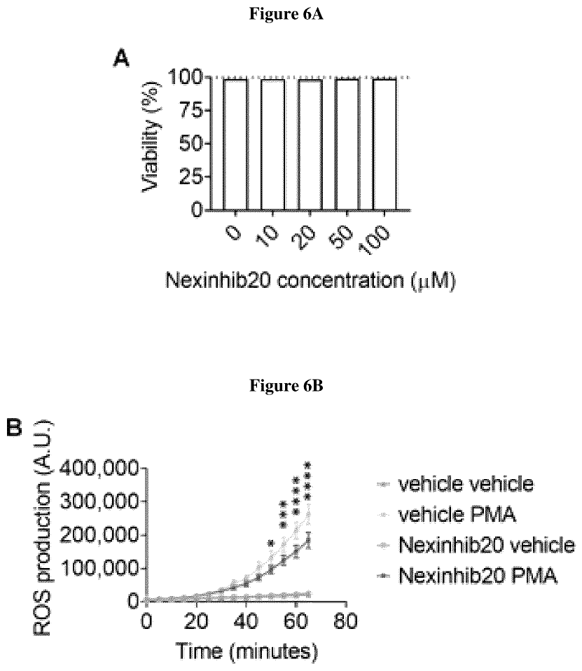

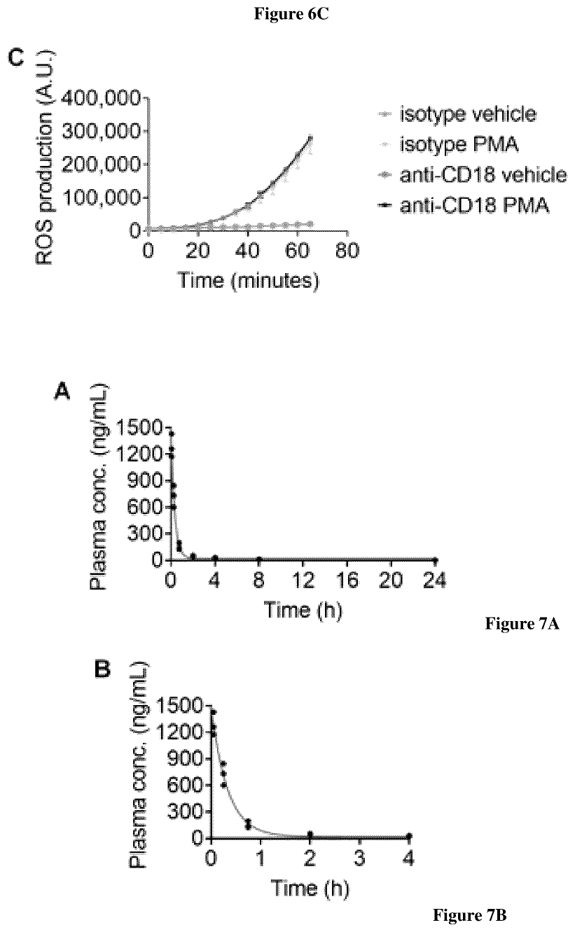

In particular, with reference to the Examples and results set forth below, Nexinhib20 was discovered to be a neutrophil exocytosis inhibitor, which was confirmed by testing β2 integrin exocytosis after IL-8 stimulation (FIG. 6B). Importantly, it was determined that Nexinhib20 also inhibited neutrophil adhesion (FIGS. 1A-1D) and β2 integrin activation (FIGS. 2A-2I) without any effect on cell viability (FIG. 6A). Thus, Nexinhib20 was confirmed as a dual-functional neutrophil inhibitor. It was further found that Rac-1 is a target of Nexinhib20 (FIGS. 3A-3H). Nexinhib20 inhibited Rac-1 activation in cells by antagonizing the Rac-1-GTP interaction with an IC50 of 29.3 μM. Since Nexinhib20 was also reported to specifically inhibit the interaction between the small GTPase Rab27a and its effector JFC1 and neutrophil exocytosis with an IC50 of 0.33 μM, it is likely that a compound of Formula 1, in particular Nexinhib20 exerts a sequential and concentration-dependent inhibition. Rab27a is critical for neutrophil exocytosis, adhesion molecule presentation, migration, and ROS production. Rac-1 is important for neutrophil integrin activation, adhesion, migration, and phagocytosis. A compound of Formula 1, in particular Nexinhib20 has the potential to work as an anti-inflammatory drug by blocking neutrophil function. Whether Rab27a or Rac-1 is more important and whether they crosstalk during Nexinhib20 inhibition of neutrophil function remains to be further investigated.

Since it has been shown that Nexinhib20 inhibits both Rab27a and Rac-1, whether a compound of Formula 1, in particular Nexinhib20 has poor specificity and may inhibit many other GTPases is of concern. All small Rab GTPases share a common mechanism of GTP-dependent binding to their respective effectors. However, each pair is characterized by highly specific binding properties, and therefore, it is unlikely that a compound of Formula 1, in particular Nexinhib20 would have a high affinity for other GTPases. In fact, the binding affinity of Nexinhib20 to Rac-1 is much lower than Rab27a as we showed an about 90-fold IC₅₀ for Rac-1 compared to Rab27a. It has also been shown that another GTPase, Rab11, was not inhibited by Nexinhib20.

It has been shown that Nexinhb20 inhibited the interaction of recombinant Rac-1 protein and GTPγS in a dose-dependent manner. This suggests that a compound of Formula I, in particular Nexinhib20, may directly bind Rac-1 and interact with key amino acids of the GTP-binding site. The crystal structure of Rac-1 complexed with a GTP analogue, guanosine-5-(βγ-imino)triphosphate (GMPPNP), has been determined. The GTP binding site includes the phosphate-binding loop residues 10-17 and residues 57 to about 61; the guanine base recognition motif residues 116-119 and 158-160; and the effector loop, residues 28-38, which interacts with the ribose and the magnesium ion. Whether a compound of Formula I, in particular Nexinhib20, directly interacts with these residues remains to be further investigated.

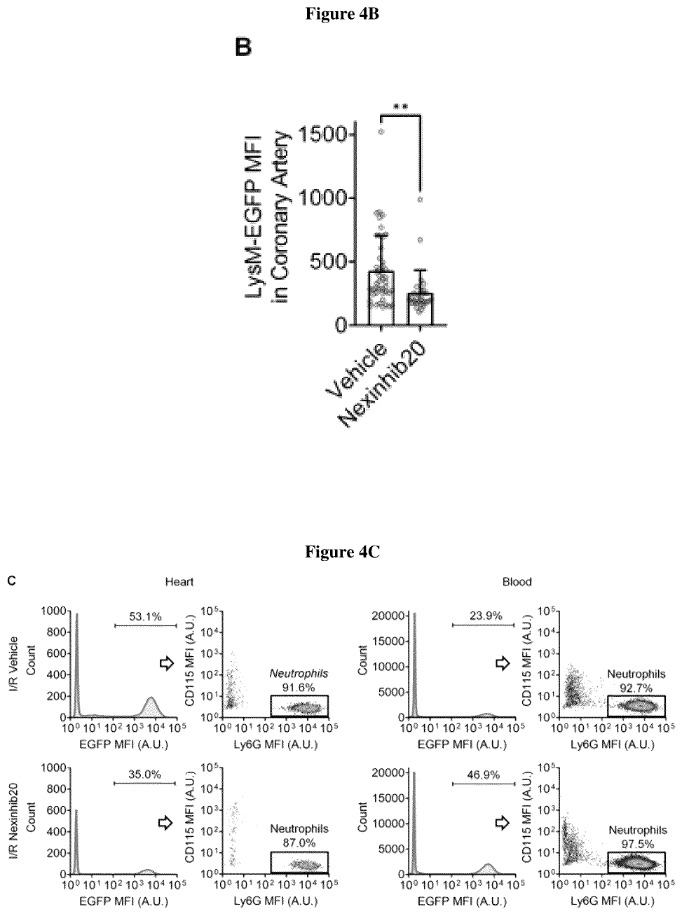

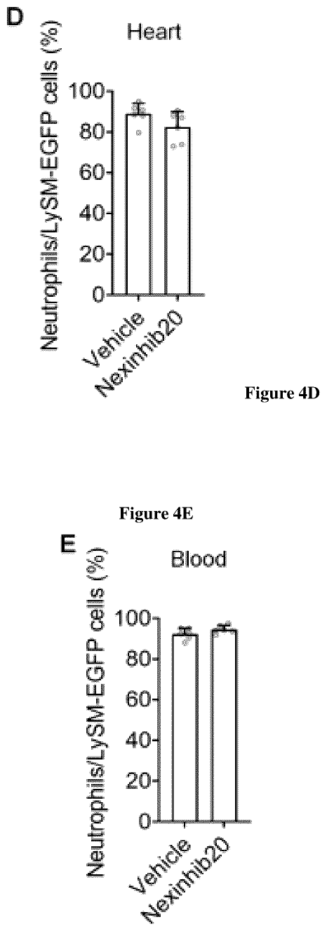

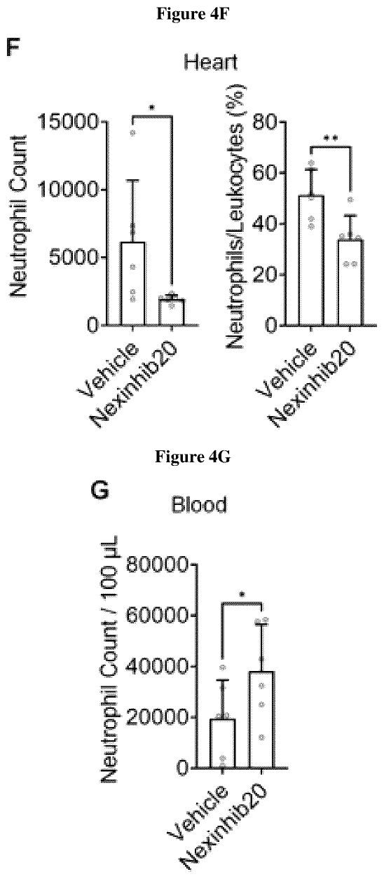

Nexinhib20 has been reported to inhibit recruitment of neutrophils to the liver and kidney in a lipopolysaccharide (LPS)-induced systemic inflammation model and to lung lumen and parenchyma in an acute LPS-induced lung injury mouse model. It is known that Nexinhib20 reduced neutrophil recruitment to the coronary artery during myocardial I/R injury (FIGS. 4A-4G), which was accomplished with multi-photon microscopy. This method can directly, and very accurately, visualize neutrophil recruitment. The multi-photon microscopy assay can also provide information in a sample with about 100 μm thickness, which is more expansive than quantification using about 8-10 μm histology sections. By combining optical clearing and light-sheet microscopy, neutrophil recruitment in the entire area at risk can be visualized and quantified. Attempts to use this method may be limited by instruments and experience with whole tissue optical clearing and staining. Another method to quantify neutrophil recruitment is flow cytometry of heart single-cell suspensions.

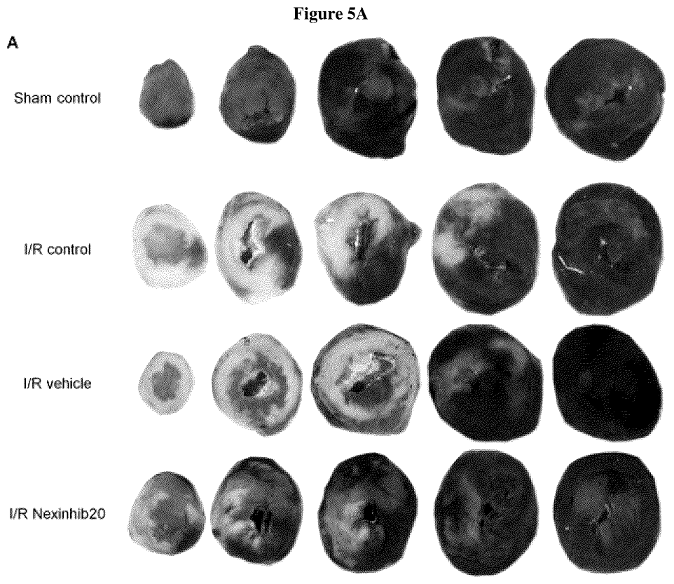

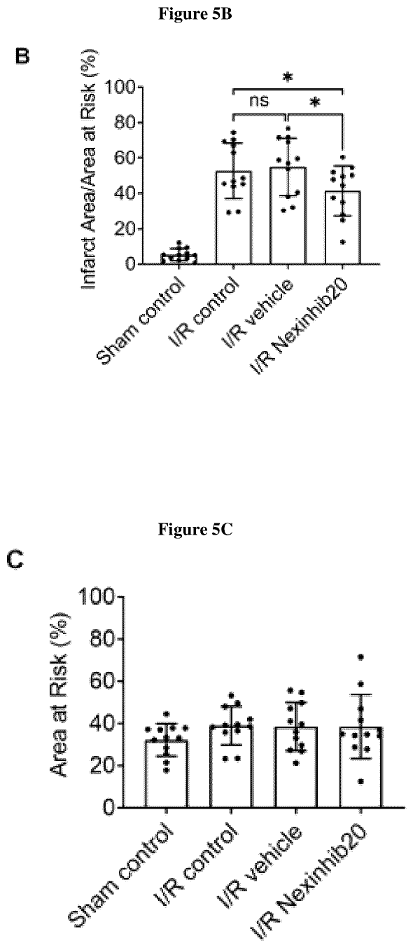

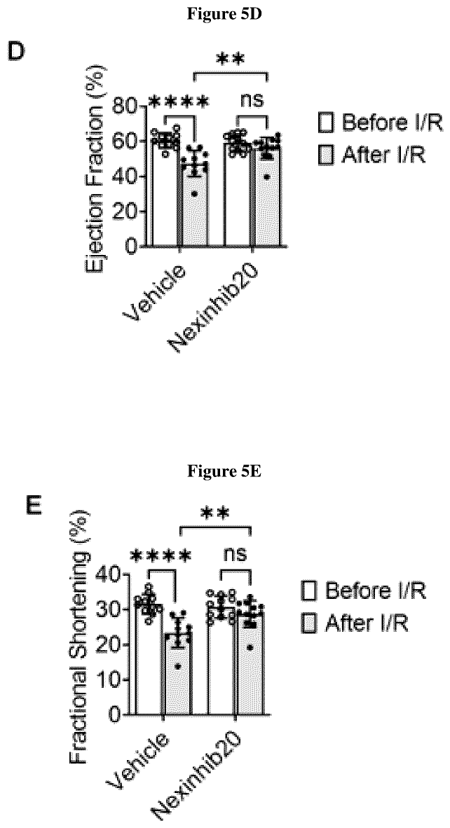

Nexinhib20 showed both anti-exocytosis and anti-adhesion activities, suggesting that a compound of Formula 1, in particular Nexinhib20 might be a dual-functional drug for myocardial I/R injury. Nexinhib20 improves myocardial I/R injury in mice by reducing infarct size by about 20% (FIGS. 5A, 5B) and almost completely restoring the left ventricle function (FIGS. 5D, 5E). Although antibodies against β2 integrins showed benefits in myocardial I/R injury in multiple species, the clinical trial using a β2 integrin antibody to treat myocardial I/R injury failed. This can be due to the long half-life of antibodies in patient circulation that also inhibits the resolution of inflammation after myocardial I/R injury, as it has been shown that accurate clearance of dead cells is a prerequisite for favorable MI healing, whereas failed resolution promotes unfavorable cardiac remodeling, which may ultimately result in heart failure. The clearance of dead cardiomyocytes and inflammatory neutrophils is orchestrated by macrophages, which are thought to derive from recruited Ly6C^{hi} monocytes, and β2 integrin antibody can block the recruitment of monocytes. Meanwhile, during the clearance of dead cells, macrophages or monocytes must migrate to dead cells and perform phagocytosis. β2 integrins are critical for both cell migration and phagocytosis. Thus, a small molecule pharmaceutical such as a compound of Formula 1, in particular Nexinhib20 that inhibits β2 integrin function for a shorter period (several hours) compared to antibodies (several weeks to months) can be an advantage in treating acute inflammatory diseases like myocardial I/R injury. This is because administering a small molecule pharmaceutical can alleviate the pro-inflammatory responses during acute inflammation and then degrade after several hours, so it will not block the later resolution of inflammation.

The pharmacokinetics of Nexinhib20 after i.p. injection on mice (FIGS. 7A-7B) has been studied, and have shown that Nexinhib20 was degraded quickly within 2 hours, which is during the acute inflammation phase. Four hours after administration, the Nexinhib20 concentration is lower than about 28.8 μg·mL⁻¹ (about 96 nM), which may not block the recruitment of monocytes/macrophages and their mediation of inflammation resolution and healing. This needs to be validated in future investigations. Furthermore, Nexinhib20-mediated inhibition of neutrophil exocytosis and ROS production (FIG. 6B) would also contribute to the attenuation of the I/R injury. Since it was demonstrated that Nexinhib20 could prevent myocardial I/R injury, a compound of Formula I, in particular Nexinhib20 can be used for other acute inflammatory diseases involving neutrophils, such as noninfectious acute lung injury, I/R injury after transplantation, ischemic stroke, systemic inflammatory response to severe injury, and multiple organ dysfunction syndrome.

In summary, a compound of Formula I, in particular Nexinhib20 have been identified as an antagonist of the Rac-1-GTP interaction. Since Rac-1 is critically involved in the functions of many cells, a compound of Formula I, in particular Nexinhib20 may be used for treatments targeting other cells. Although this study focused on myocardial I/R injury, it is important to discuss the role of Rac-1 in cardiomyocytes. Rac-1 is not only important for leukocyte activation and ROS production but it is also essential for ROS production by cardiomyocytes. Cardiomyocyte-specific overexpression of an active Rac mutation aggravated myocardial I/R injury, and myocardial I/R-induced ventricular arrhythmia was significantly decreased in cardiac-specific Rac-1 knockdown mice. Another Rac-1 inhibitor, NSC23766, decreased I/R-induced ventricular arrhythmia. Active Rac-1 was upregulated in failing myocardium of patients with ischemic cardiomyopathy and dilated cardiomyopathy. Statin treatment decreased myocardial Rac1-GTPase activity. Rac-1 activation was involved in the hypertrophic response of cardiomyocytes, hyperglycemia-induced apoptosis of cardiomyocytes in diabetes, and doxorubicin-induced cardiotoxicity. Besides cardiomyocytes, shear stress-induced Rac-1 activation in endothelial cells is responsible for ICAM-1 expression, which is critical for the recruitment of neutrophils and inflammatory responses. Inhibition of Rac1 GTPase decreases vascular oxidative stress, improves endothelial function, and attenuates atherosclerosis development in mice. Overall, most studies supported that inhibition of Rac-1 was beneficial to most cardiomyopathies, therefore, Rac-1-specific inhibitors, such as NSC23766 or statin may help as well. Chemical modulation may also help to increase the affinity of Nexinhib20 to Rac-1 that increases inhibition efficiency.

Neutrophil-mediated tissue damage after I/R injury is a multifactorial process that depends on β2 integrin-dependent neutrophil adhesion, recruitment, and secretion. A compound of Formula I, in particular Nexinhib20, in addition to exocytosis, can also inhibit human neutrophil adhesion and β2 integrin activation by targeting Rac1 GTPase. A compound of Formula I, in particular Nexinhib20 can decrease neutrophil recruitment in vivo and decreased infarct size after mouse myocardial I/R injury, further validating that inhibition of neutrophil recruitment and activation increases the likelihood of a favorable outcome during myocardial tissue damage.

In an aspect, a compound of Formula I, in particular Nexinhib20, is an antagonist of the of the Rac-1-GTP interaction. The compound of Formula I, in particular Nexinhib20, can have an IC₅₀ (the concentration at which a substance exerts half of its maximal inhibitory effect) of 100 nM to 500 μM, or 500 nM to 250 μM, or 1 μM to 100 μM with respect to the Rac-1-GTP interaction. The compound of Formula I, in particular Nexinhib20, can have an IC₅₀ of 100 nM to 500 μM, or 500 nM to 250 μM, or 1 μM to 100 μM with respect to Rab27a. The compound of Formula I, in particular Nexinhib20, can have an IC₅₀ of 100 nM to 500 μM, or 500 nM to 250 μM, or 1 μM to 100 μM with respect to Rac-1. In an aspect, the compound of Formula 1, in particular Nexinhib20 has improved specificity for GTPases other than Rab27a and/or Rac-1. In an aspect, a compound of Formula I, in particular Nexinhib20, has poor specificity or no affinity for other GTPases, in particular, Rab11.

Specific methods for synthesis of compounds of Formula 1, Formula 1a, and Nexinhib20 are known to those of ordinary skill in the art.

In an embodiment, a composition for treating or preventing a neutrophil-related acute inflammatory condition (e.g., an acute inflammatory disease involving neutrophils) in a subject in need thereof includes a composition comprising a compound of Formula 1 or Formula 1a, in particular Nexinhib 20, and/or a pharmaceutically acceptable salt and/or a formulation thereof. Exemplary neutrophil-related acute inflammatory conditions or diseases include those such as myocardial ischemia-reperfusion injury, noninfectious acute lung injury, ischemia-reperfusion injury after transplantation, ischemic stroke, systemic inflammatory response to severe injury, multiple organ dysfunction syndromemyocardial ischemia-reperfusion injury, or the like.

In another embodiment, a pharmaceutical composition for treating or preventing myocardial ischemia-reperfusion injury in a subject includes a compound of Formula 1 or Formula 1a, in particular Nexinhib 20, and/or a pharmaceutically acceptable salt and/or a formulation thereof. In an embodiment, the subject is a mammal, for example a human.

In still another embodiment, a pharmaceutical composition for treating or preventing myocardial ischemia-reperfusion injury in a subject includes Nexinhib 20 and/or a pharmaceutically acceptable salt and/or a formulation thereof. In an embodiment, the subject is a mammal, for example a human.

The compound of Formula 1 or Formula 1a, in particular Nexinhib 20, and/or a pharmaceutically acceptable salt and/or formulation thereof can be formulated with an adjuvant to provide the pharmaceutical composition. Suitable adjuvants depend on the delivery method and form, and are described in more detail below.

A method for treating or preventing a neutrophil-related acute inflammatory condition (e.g., an acute inflammatory disease involving neutrophils) in a subject in need thereof includes providing a pharmaceutical composition including a compound of Formula 1, in particular Nexinhib 20, and/or a pharmaceutically acceptable salt and/or formulation thereof; and administering the pharmaceutical composition to the subject. The subject may be a mammal, and in an aspect, the subject is human. Exemplary neutrophil-related acute inflammatory conditions or diseases include those such as myocardial ischemia-reperfusion injury, noninfectious acute lung injury, ischemia-reperfusion injury after transplantation, ischemic stroke, systemic inflammatory response to severe injury, multiple organ dysfunction syndromemyocardial ischemia-reperfusion injury, or the like.

In another embodiment, a method for treating or preventing myocardial ischemia-reperfusion injury in a subject includes: providing a pharmaceutical composition including a compound of Formula 1 or Formula 1a, in particular Nexinhib 20, and/or a pharmaceutically acceptable salt and/or formulation thereof; and administering the pharmaceutical composition to the subject. The subject may be a mammal, and in an aspect, the subject is human.

In still another embodiment, a method for treating or preventing myocardial ischemia-reperfusion injury in a subject includes: providing a pharmaceutical composition including Nexinhib 20 or a pharmaceutically acceptable salt thereof; and administering the composition to the subject. The subject may be a mammal, and in an aspect, the subject is human.

The pharmaceutical composition including a compound of Formula 1 or Formula 1a, in particular Nexinhib 20, and/or a pharmaceutically acceptable salt and/or formulation thereof can be administered during the acute phase of stroke, between the time the stroke occurs and lasting for up to about 7 days after stroke. In an aspect, administration of the pharmaceutical composition including a compound of Formula 1, in particular Nexinhib 20, and/or a pharmaceutically acceptable salt and/or formulation thereof, is ceased after the acute phase of stroke, i.e., after 7 days post-stroke. In another aspect, the pharmaceutical composition including a compound of Formula 1, in particular Nexinhib 20, and/or a pharmaceutically acceptable salt and/or formulation thereof, is administered in the acute phase, but ceased 1 day post-stroke, 2 days post-stroke, 3 days post-stroke, 4 days post-stroke, 5 days post-stroke, 6 days post-stroke, or 7 days post-stroke. In another aspect, the pharmaceutical composition including a compound of Formula 1, in particular Nexinhib 20, and/or a pharmaceutically acceptable salt and/or formulation thereof, is administered in the acute, the subacute, the chronic phase of stroke, or a combination thereof. Administration during the chronic phase of stroke is expected to be beneficial.

In an aspect, administering a pharmaceutical including a compound of Formula 1 or Formula 1a, in particular Nexinhib 20, and/or a pharmaceutically acceptable salt and/or formulation thereof, can be by oral administration, for example, administration of a solid or liquid oral pharmaceutical formulation.

In another aspect, administering a pharmaceutical composition including a compound of Formula 1 or Formula 1a, in particular Nexinhib 20, and/or a pharmaceutically acceptable salt and/or formulation thereof, can be by intravenous injection, such as injection into the general circulation or by targeted infusion whereby the agent is slowly supplied close to the site of the blockage that triggered the stroke. Infusion can be via an endovascular catheter such as a catheter ready to be used, being used, or having been used in providing a thrombolytic therapeutic to the subject; or a catheter having been used in conjunction with a procedure on the subject involving use of a clot-removal device.

A pharmaceutical composition c including a compound of Formula 1 or Formula 1a, in particular Nexinhib 20, and/or a pharmaceutically acceptable salt and/or formulation thereof, can be administered one minute to up to 3 hours before administering a thrombolytic therapeutic or clot retrieval mechanically via an endovascular approach (also known as mechanical lysis) to the subject. A pharmaceutical composition including a compound of Formula 1 or Formula 1a, in particular Nexinhib 20, and/or a pharmaceutically acceptable salt and/or formulation thereof, can be administered concomitantly with a thrombolytic therapeutic or clot retrieval mechanically via an endovascular approach to the subject. Alternatively, a pharmaceutical composition including a compound of Formula 1 or Formula 1a, in particular Nexinhib 20, and/or a pharmaceutically acceptable salt and/or formulation thereof, can be administered after a thrombolytic therapeutic or clot retrieval mechanically via an endovascular approach to the subject. Thrombolytic therapeutics include compounds such as aspirin, clopidogrel, triclopidine, tissue plasminogen activator, urokinase, and streptokinase. A combination thereof can be used.

For oral administration, the pharmaceutical composition can be in liquid form, for example, solutions, syrups, or suspensions, or can be presented as a drug product for reconstitution with water or other suitable vehicle before use. Such liquid preparations can be prepared by conventional means with pharmaceutically acceptable additives (adjuvants) such as suspending agents (e.g., sorbitol syrup, cellulose derivatives, or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical composition can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e. g., magnesium stearate, talc or silica); disintegrants (e.g., potato starch or sodium starch glycolate); or wetting agents (e.g., sodium lauryl sulphate). The tablets can be coated by methods well-known in the art.

The pharmaceutical composition for oral administration can be suitably formulated to give controlled release of the active compound.

For buccal administration, the pharmaceutical composition can take the form of tablets or lozenges formulated in conventional manner.

For administration by inhalation, the pharmaceutical composition can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can be formulated for parenteral administration by injection, e.g., by bolus injection or continuous infusion via either intravenous, intraperitoneal, or subcutaneous injection. Many of the injectable formulations have their own specific co-solvents or excipients, which may or may not be in addition to the salts that conjugate with the drug substance. Pharmaceutical compositions for injection can be presented in unit dosage form, e.g., in ampoules or in multi-dose containers, with an added preservative. The pharmaceutical composition can take such forms as suspensions, solutions, or emulsions in oily or aqueous vehicles, and can contain formulatory agents such as suspending, stabilizing, or dispersing agents, or a combination thereof. Alternatively, a compound of Formula 1, in particular Nexinhib 20, and/or a pharmaceutically acceptable salt and/or formulation thereof, can be in powder form for constitution with a suitable vehicle, e.g., sterile pyrogen-free water, before use.

The pharmaceutical composition can also be formulated as a depot preparation. Such long-acting formulations can be administered by implantation (e.g., subcutaneously or intramuscularly) or by intramuscular injection. Thus, for example, the pharmaceutical composition can be formulated with suitable polymeric or hydrophobic materials (e.g., as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The pharmaceutical composition can, if desired, be presented in a pack or dispenser device, which can contain one or more unit dosage forms containing the active ingredient. The pack can for example include metal or plastic foil, such as a blister pack. The pack or dispenser device can be accompanied by instructions for administration.

The amount of a compound of Formula 1 or Formula 1a, in particular Nexinhib 20, and/or a pharmaceutically acceptable salt and/or formulation thereof that can be combined with pharmaceutically acceptable adjuvant to produce a single dosage form can vary depending upon the host treated and the particular mode of administration. The specific therapeutically effective amount for a particular patient will depend on a variety of factors, including the activity of the specific compound employed, the age, body weight, general health, sex, diet, time of administration, route of administration, rate of excretion, drug combination, and the severity of the particular disease undergoing therapy. In some instances, dosage levels below the lower limit of the aforesaid range can be more than adequate, while in other cases still larger doses can be used without causing any harmful side effects, provided that such higher dose levels are first divided into several small doses for administration throughout the day. The concentrations of a compound of Formula 1 or Formula 1a, in particular Nexinhib 20, and/or a pharmaceutically acceptable salt and/or formulation thereof in therapeutic compositions will vary depending upon a number of factors, including the dosage of the drug to be administered, the chemical characteristics (e.g., hydrophobicity) of the particular compound employed, and the route of administration. In an aspect, a pharmaceutical composition including a compound of Formula 1, in particular Nexinhib 20, and/or a pharmaceutically acceptable salt and/or formulation thereof, is administered at a dosage of about 0.05 mg/kg to about 0.5 mg/kg to about 5 mg/kg of body weight of the subject.

The invention is further illustrated by the following non-limiting Examples.

### EXAMPLES

### Materials and Methods

### Reagents

Recombinant human P-selectin-Fc, ICAM-1-Fc, and IL-8 were purchased from R&D Systems. The Alexa Fluor 488 (AF488)-conjugated and unconjugated conformation-specific monoclonal antibodies mAb24 to human β2-I-like-domain (which reports the headpiece-opening), unconjugated mouse anti-human CD18 mAb (blocking, clone TS1/18), AF594-conjugated rat anti-mouse CD31 mAb, allophycocyanin (APC)-conjugated rat anti-mouse CD115 mAb, PE-conjugated rat anti-mouse Ly6G mAb, AF700-conjugated rat anti-mouse CD45 mAb, unconjugated mouse IgG1 isotype control, APC-conjugated rat anti-mouse IgG1 secondary mAb, and Zombie Yellow Fixable Viability Kit were purchased from Biolegend. The KIM127 mAb to human β2-IEGF-domain, which reports the ectodomain extension, was purified at the Lymphocyte Culture Center at the University of Virginia from hybridoma supernatant (American Type Culture Collection). KIM127 was directly labeled by DyLight 550 (DL550) using DyLight antibody labeling kits from Thermo Fisher Scientific. Nexinhib20 was purchased from Tocris. Casein blocking buffer, Fluo-4 AM, and Pierce protease inhibitor mini-tablets were purchased from Thermo Fisher Scientific. Ghost Dye Blue 516 was purchased from Tonbo Biosciences. Polymorphprep was purchased from Accurate Chemical. Roswell Park Memorial Institute (RPMI) medium 1640 without phenol red and phosphate-buffered saline (PBS) were purchased from Gibco. Human serum albumin (HSA) and fetal bovine serum (FBS) were purchased from Gemini Bio Products. Formalin and non-fat milk were purchased from Fisher Scientific. The Rac-1 Activation Assay Biochem Kit, which contains PAK-PBD protein beads, purified His-tagged Rac-1 protein, GTPγS (non-hydrolysable GTP analog), GDP, and several buffers, were purchased from Cytoskeleton, Inc. A bulk custom order of purified His-tagged Rac-1 protein was purchased from Quintarabio. N-formylmethionyl-leucyl-phenylalanine (fMLP), triphenyl tetrazolium chloride (TTC), Phorbol-12-myristate-13-acetate (PMA), polybrene, paraformaldehyde (PFA), and dimethyl sulfoxide (DMSO) were purchased from Sigma Aldrich. 2× Laemmli sample buffer and Mini-PROTEAN TGX precast gels were purchased from BioRad. Mouse monoclonal anti-Rac-1 antibody was purchased from BD Biosciences. Horseradish peroxidase (HRP)-conjugated horse anti-mouse antibody was purchased from Cell Signaling Technology. Trappsol (2-Hydroxypropyl-β-cyclodextrin) was purchased from Cyclodextrins CTD, Inc. Enhanced Chemiluminescence (ECL) Ultra was purchased from Lumigen. Penicillin, streptomycin, and amphotericin B solutions were purchased from Hyclone. The total ROS Assay kit was purchased from Invitrogen.

### Human Neutrophil Isolation

Heparinized whole blood samples were obtained from healthy human donors after informed consent, as approved by the Institutional Review Board of the La Jolla Institute for Immunology in accordance with the Declaration of Helsinki. Informed consent was obtained from all donors. Neutrophils were isolated using a Polymorphprep (a mixture of sodium metrizoate and Dextran 500) density gradient. Briefly, human blood was applied to Polymorphprep, centrifuged at 500 g for 35 minutes at 20-25° C., resulting in neutrophils concentrated in a layer between peripheral blood mononuclear cells and erythrocytes. After washing with PBS twice, the neutrophils (>95% purity by flow cytometry, no visible activation by microscopy) were resuspended in RPMI-1640 without phenol red plus 2% HSA and used within four hours. Neutrophils were incubated with FcR blocking reagents for 10 minutes at room temperature (RT) before all the experiments.

### Microfluidic Device

The assembly of the microfluidic devices used in this study and the coating of coverslips with recombinant human P-selectin-Fc and ICAM-1-Fc with or without IL-8 have been described previously. Briefly, coverslips were coated with P-selectin-Fc (2 μg·ml⁻¹) and ICAM-1-Fc (10 μg·ml⁻¹) without or with IL-8 (10 μg·ml⁻¹) for two hours and then blocked for one hour with casein (1%) at RT. After coating, coverslips were sealed with polydimethylsiloxane chips by magnetic clamps to create flow chamber channels ˜29 μm high and ˜300 μm across. By modulating the pressure between the inlet well and the outlet reservoir, 6 dyn·cm⁻² wall shear stress was applied in all experiments.

### Microfluidic Perfusion Assay

To study the rolling and arrest of neutrophils, isolated human neutrophils (5×10⁶ cells·ml⁻¹) were perfused in the microfluidic device over a substrate of recombinant human P-selectin-Fc and recombinant human ICAM-1-Fc with or without IL-8 under a shear stress of 6 dyn·cm⁻². Neutrophils were incubated with Nexinhib20 (10 μM) or vehicle (DMSO) for one hour at RT before being perfused into the microfluidic devices. Time-lapse images (one frame per second) were taken by an IX71 inverted research microscope (Olympus America) with a 40× NA 0.9 air objective during the perfusion to quantify rolling velocity. The quantification was done using the “Manual tracking” plugin in FIJI-ImageJ v2.0. Cell tracks (FIG. 1A) and rolling velocity were obtained (FIGS. 1B, 2C). After perfusion with neutrophils for 10 minutes, the microfluidic device was washed with RPMI-1640 without phenol red plus 2% HSA for 5 minutes. Then, the arrested neutrophils were counted in nine fields-of-view per group (FIG. 1D).

### Mice

C57BL/6J wild-type mice (000664; JAX) were originally obtained from the Jackson Laboratory. LysM-EGFP or Lyz2-EGFP mice were originally obtained from Albert Einstein College of Medicine through a material transfer agreement. Mice were fed a standard rodent chow diet and were housed in microisolator cages in a pathogen-free facility in the Center for Comparative Medicine at UConn Health. All experiments followed the UConn Health Institutional Animal Care and Use Committee (IACUC) guidelines, and approval for the use of rodents was obtained from the UConn Health IACUC according to criteria outlined in the Guide for the Care and Use of Laboratory Animals from the National Institutes of Health. Both male and female mice aged from 12 to 16 weeks were used in the experiments.

### Ischemia-Reperfusion Injury

Mice were subjected to 35 minutes of myocardial ischemia and 1 (for multi-photon microscopy and flow cytometry) or 22-26 (for TTC-phthalo-blue staining) hours of reperfusion. The reason we use two time points to harvest is because neutrophil recruitment happens 1 hour after the reperfusion, and the infarct size can be significantly quantified by TTC-phthalo-blue staining after about 24-hour reperfusion. Briefly, anesthesia was induced with an intraperitoneal injection of ketamine hydrochloride (125 mg·kg⁻¹) and xylazine (12.5 mg·kg⁻¹). Mice were intubated with 24G×3/4″ Surflo i.v. catheter and ventilated using MiniVent 845 (Harvard Apparatus).

Surgeries were performed under an SMZ168 Stereo Zoom microscope (Motic). Ischemia was achieved by ligating the left anterior descending coronary artery (LAD) using a 6-0 silk suture with a section of PE-10 tubing placed over the LAD, 1 mm from the tip of the normally positioned left atrium. One important problem in drug administration is water solubility, which greatly affects drug absorption and bioavailability. In our study, we used Trappsol (2-hydroxypropyl-β-cyclodextrin) as a cosolvent for in vivo administration to increase Nexinhib20 solubility. In clinics, the primary percutaneous coronary intervention aims to be performed less than 90 min (within 60 min is preferable) after the patient arrives. To mimic a prevention treatment of reperfusion injury before the primary percutaneous coronary intervention, which is feasible in the clinics, Nexinhib20 (100 mM, 10 μL in DMSO mixed with 190 μL 10% Trappsol per mouse) or vehicle control were administered i.p. 30 minutes before the reperfusion. After occlusion for 35 minutes, reperfusion was initiated by releasing the ligature and removing the PE-10 tubing. The chest wall was closed, the animal extubated, and body temperature was maintained by use of a 37° C. warm pad. Hearts were harvested 1 or 22-26 hours later. The loosened suture was left in place and then retied for the purpose of evaluating the ischemic area. Sham control and no drug administered control were performed as well.

### Flow Cytometry

Isolated human neutrophils (2×10⁶ cells·mL⁻¹ in RPMI-1640 without phenol red plus 2% HSA) were incubated with Nexinhib20 (10 μM) or vehicle (DMSO) for one hour at RT before being assayed. To monitor the dynamics of β2 integrin activation, 400 μL of 2.5×10⁵ cells·mL⁻¹ neutrophils were assessed by an LSRII analyzer (BD Biosciences, San Jose, CA) for 10 s. After adding 0.5 μg·mL⁻¹ AF488-conjugated mAb24 and DL550-conjugated KIM127 (final concentration), cells were put back into the analyzer for another 5 minutes. Then, after adding 1 μg·mL⁻¹ IL-8, cells were put back into the analyzer for another 10 minutes. The curves showing the dynamics of integrin activation (FIGS. 2A, 2B) were generated by FlowJo software (version 10.6). The antibody specificities were validated in our previous study using β2 integrin knockout cells and β2-integrin-activation-deficient talin-1 knockout cells. Compensations were performed before all experiments.

To quantify the percentage of mAb24 and KIM127 epitopes and assess inhibition of β2 integrin exocytosis, pan-CD18 mAb24 TS1/18, which has the same isotype (mouse IgG1) as mAb24 and KIM127, was used. Isolated human neutrophils (5×10⁵ cells·mL⁻¹ in RPMI-1640 without phenol red plus 2% HSA) were incubated with Nexinhib20 (10 μM) or vehicle (DMSO) for one hour at RT before being assayed. Neutrophils were mixed with unconjugated mAb24 (1 μg·mL⁻¹), KIM127 (1 μg·mL⁻¹), TS1/8 (1 μg·mL⁻¹), or mouse IgG1 isotype control (1 μg·mL⁻¹), and incubated with 1 μg·mL⁻¹ IL-8 at RT for 10 min. After incubation, neutrophils were fixed by 1% PFA at 4° C. for 10 min. After two washes with PBS, cells were incubated with APC-conjugated rat anti-mouse IgG1 secondary mAb (1 μg·mL⁻¹) at RT for 10 min. After two washes with PBS, cell fluorescence was assessed with an LSRII (BD Biosciences, San Jose, CA) and analyzed with FlowJo software (version 10.6). The quantifications of mAb24, KIM127, TS1/18, and isotype mean fluorescence intensities (MFI) (FIGS. 2C-2E) were analyzed by FlowJo software (version 10.6) and obtained from six replicates. MFI of isotype controls was subtracted as background signal. Since mAb24, KIM127, and TS1/18 are all IgG1 isotypes and we used the same secondary antibody, the percentage of high-affinity and extended β2 integrins (FIGS. 2F and 2G) can be calculated by dividing the MFI of mAb24 and KIM127 by the MFI of TS1/18.

To monitor the dynamics of intracellular calcium (Ca²⁺) flux, neutrophils (2×10⁶ cells·mL⁻¹ in RPMI-1640 without phenol red plus 2% HSA) were incubated with Fluo-4 (4 μg·mL⁻¹) for one hour at RT. After washes, neutrophils were resuspended in RPMI-1640 without phenol red plus 2% HSA and assessed by an LSRII analyzer (BD Biosciences, San Jose, CA). One minute after analyzing, 1 μg·mL⁻¹ IL-8 was added to the cells. Cells were put back into the analyzer for another 9 minutes. The curves showing the dynamics of intracellular Ca²⁺ flux (FIG. 2H) were generated by FlowJo software (version 10.6). The quantification of Fluo-4 MFI (FIG. 2I) was analyzed by FlowJo software (version 10.6) and obtained from three individual experiments.

To assess the viability of neutrophils (FIG. 6B), neutrophils (2×10⁶ cells·mL⁻¹ in RPMI-1640 without phenol red plus 2% HSA) were incubated with different concentrations (0, 10, 20, 50, and 100 μM) of Nexinhib20 at RT for one hour. After washes, neutrophils were incubated with Ghost Dye Blue 516 at RT for 15 minutes. After washes, cell fluorescence was assessed with an LSRII (BD Biosciences, San Jose, CA) and analyzed with FlowJo software (version 10.6).

To assess the neutrophil recruitment in myocardial I/R injury, LysM-EGFP mice underwent 35 minutes of ischemia and 1 hour of reperfusion. To mimic a prevention treatment of reperfusion injury before the primary percutaneous coronary intervention, which is feasible in the clinics, Nexinhib20 (100 mM, 10 μL per mouse) or vehicle control was administered i.p. 30 minutes prior to the reperfusion. After the 1-hour reperfusion, the mouse heart was harvested and perfused with ice-cold PBS to remove residual blood and unbound leukocytes, transferred into an ice-cold gentleMACS C tube, cut into about 1 mm³ pieces, suspended with 5 mL PBS plus 2% FBS, 2 mM EDTA, and 0.08 μg·mL⁻¹ APC-conjugated anti-CD115 mAb, and homogenized five times by the ‘m_Heart_01’ program of the gentleMACS Dissociator (Miltenyi). The cell suspension was filtered by 70 μm nylon mesh strainer (Fisher), centrifuged at 500×g, 4° C. for 5 minutes, resuspended in 200 μL 1:300 diluted Zombie Yellow fixable viability dye, and incubated on ice for 15 minutes. After centrifuging at 500×g, 4° C. for 5 minutes, cells were resuspended in 200 μL ice-cold PBS containing 1.25 μg·mL⁻¹ AF700-conjugated anti-CD45 mAb and 1 μg·mL⁻¹ PE-conjugated anti-Ly6G mAb and incubated on ice for 10 minutes. After being fixed with 1% PFA and washes with ice-cold PBS, cell fluorescence was assessed with an LSRII (BD Biosciences, San Jose, CA) and analyzed with FlowJo software (version 10.6).

Peripheral blood of the above mice was also collected. 100 μL was mixed with 200 μL 1:300 diluted Zombie Yellow fixable viability dye and incubated on ice for 15 minutes. After centrifuging at 500×g, 4° C. for 5 minutes, cells were resuspended in 200 μL ice-cold PBS containing 1.25 μg·mL⁻¹ AF700-conjugated anti-CD45 mAb, 1 μg·mL⁻¹ PE-conjugated anti-Ly6G mAb, and 2 μg·mL⁻¹ APC-conjugated anti-CD115 mAb, and incubated on ice for 10 minutes. After being fixed with 1% PFA, red blood cells were lysed with deionized water for 30 seconds (stopped by adding 10×PBS). Leukocyte fluorescence was assessed with an LSRII (BD Biosciences, San Jose, CA) and analyzed with FlowJo software (version 10.6).

### Reactive Oxygen Species (ROS) Production

ROS production of isolated human neutrophils was quantified by using the Total ROS Assay kit from Invitrogen. A black, clear bottom, non-treated 96-well plate was used in this assay. Before the assay, the 96-well plate was coated with 10 μg·mL⁻¹ human ICAM-1-Fc at RT for 2 hours and washed twice with PBS. Isolated human neutrophils (2×10⁶ cells·mL⁻¹) were incubated with Nexinhib20 (10 μM) or vehicle (DMSO) for one hour at RT before being assayed. After centrifuging at 300×g, RT for 2 min, cells were resuspended at 10⁶ cells·mL⁻¹ in the ROS Assay Stain Solution from the kit and incubated with 2 μg·mL⁻¹ mouse anti-human CD18 blocking mAb (TS1/18 to block neutrophil adhesion) or isotype control at RT for 10 min. The 100 μL·well⁻¹ neutrophils (3 replicates per group) were seeded into the ICAM-1-coated 96-well plate. The background ROS before stimulation was measured by Cytation 1 Cell Imaging Multi-Mode Reader (Filter set: Green, Ex: 485/20 nm, Em: 528/20 nm, BioTek, Santa Clara, CA). Then 100 nM PMA was added to each well, and ROS production was measured by Cytation 1 Cell Imaging Multi-Mode Reader every 5 minutes.

### Cell Culture

The HL60 cells and CXCR2-expressing HL60 cells (HL60-CXCR2)(50) were gifts from Dr. Orion D. Weiner at the University of California San Francisco and Dr. Ann Richmond at the Vanderbilt University School of Medicine, respectively. HL60-CXCR2 cells were selected with G418 (0.5 μg·mL⁻¹) to maintain CXCR2 expression. Cells were maintained in culture medium (RPMI-1640, 10% FBS, 100 μg·mL⁻¹ penicillin, 100 μg·mL⁻¹ streptomycin, and 250 ng·mL⁻¹ amphotericin B) at 37° C. and 5% CO₂. In most experiments, cells were differentiated with 1.3% DMSO for 7 days before assays. Cells were checked monthly for mycoplasma infection using the e-Myco plus Mycoplasma PCR Detection Kit.

### Rac-1-GTP Pull-Down and Western Blots

Differentiated HL60 or HL60-CXCR2 cells or isolated human neutrophils (2×10⁶ cells·mL⁻¹ in RPMI-1640 without phenol red plus 2% HSA) were incubated with Nexinhib20 (10 μM) or vehicle (DMSO) for one hour at RT. After washes, cells were resuspended in RPMI-1640 without phenol red (10⁷ cells·mL⁻¹) and incubated with or without stimulators (100 nM fMLP for HL60, 1 μg·mL⁻¹ IL-8 for HL60-CXCR2) at RT for 1 minute. Cells were lysed by 1:1 addition of 2× Triton X-100 lysis buffer (final concentration: 1% Triton X-100, 50 mM HEPES pH 7.0, 150 mM NaCl, 10% glycerol, 1.5 mM MgCl₂, 1 mM EGTA, 1 mM sodium orthovanadate, 1 mM phenylmethylsulfonyl fluoride, plus protease inhibitor mixture—one Pierce protease inhibitor mini-tablet per 5 mL 2× buffer) on ice for 5 minutes. After centrifuging at 16,000×g, 4° C. for 8 minutes, supernatants were saved as protein samples.

For the in vitro Rac-1/GTP binding competition assays, purified His-tagged Rac-1 (0.08 μg·mL⁻¹ in 1× Triton X-100 lysis buffer) were mixed with loading buffer (from the Rac-1 activation assay biochem kit, 1:10) and Nexinhib20 (1, 3, 10, 30, 100, 300, and 1000 μM) or vehicle (DMSO). Then samples were incubated with GTPγS (0.4 μM) or GDP (0.8 mM) at RT for 15 minutes. The reaction was stopped by transferring samples to 4° C. and adding the stop buffer (from the Rac-1 activation assay biochem kit, 1:10).

The Rac-1-GTP pull-down was performed using Rac-1 activation assay biochem kit following manufacturer's instructions. Briefly, protein samples were immediately incubated with p21 activated kinase 1-p21 binding domain (PAK-PBD) beads (10 μL per 1 mL sample) for one hour at 4° C. Then beads were pelleted by centrifugation at 5000×g, 4° C. for 8 minutes. After removal of most of the supernatant, beads were washed twice with 500 μL washing buffer from the kit. Beads were resuspended with 2× Laemmli sample buffer and boiled for two minutes.

Protein samples (before and after the Rac-1-GTP pull-down) were separated using sodium dodecyl sulfate-polyacrylamide gel electrophoresis (SDS-PAGE) and transferred onto nitrocellulose membranes. Membranes were blocked for about 30 min in tris-buffered saline with 0.1% tween 20 (TBST) plus 5% non-fat milk. After blocking, membranes were incubated overnight with mouse monoclonal anti-Rac-1 antibody diluted 1:2000 in TBST at 4° C., and HRP-conjugated horse anti-mouse antibody diluted 1:5000 in TBST plus 5% non-fat milk at RT for one hour. ImageQuant LAS 4000 (GE) was used to image membranes after adding ECL Ultra (FIGS. 3A-3H).

### Multi-Photon Microscopy

Mice underwent 35 minutes of ischemia and 1 hour of reperfusion. To mimic a prevention treatment of reperfusion injury before the primary percutaneous coronary intervention, which is feasible in the clinics, Nexinhib20 (100 mM, 10 μL per mouse) or vehicle control were administered i.p. 30 minutes prior to the reperfusion. After the reperfusion, the mouse heart was harvested and perfused with PBS to remove residual blood and unbound leukocytes and was incubated with anti-CD31-AF594 mAb (10 μg/mL, 250 μL per heart) to label the coronary artery sequentially. The explanted heart was immersed in PBS and imaged by a multi-photon microscope immediately. The Bruker's upright multi-photon microscope (#4269) was equipped with a Mai Tai High-Performance Ti:sapphire femtosecond pulsed laser (tuning range 690-1020 nm, set to 780 nm excitation in this assay) and a 20× NA 0.95 water immersion objective. The bandpass filters in front of the corresponding four different photomultiplier tube detectors are 660/40, 595/50, 525/50, and 460/50 nm. The 595/50 nm channel and 525/50 nm channel were used for EGFP and AF594 imaging, respectively. Three-dimensional z-stack series (5 μm interval, 10-20 stacks) images of the coronary artery were acquired (FIG. 4A). The mean fluorescence intensity of EGFP within the coronary artery was quantified by FIJI-ImageJ v2.0.

### TTC-Phthalo-Blue Staining

To assess the ischemic area at risk after 22-26 hours of reperfusion, hearts were excised, infused with PBS and freshly prepared 10% phthalo-blue (PBS with 0.75% tween 20) through the aorta and coronary arteries in a retrograde fashion, frozen at −20° C. for 10 minutes, and sliced into five to six 1 mm cross-sections with the aid of a pre-freeze acrylic matrix (ZIVIC Labs). The heart sections were incubated with freshly prepared 1% TTC solution (Sigma-Aldrich) at 37° C. for 10 minutes and fixed with formalin. Viable myocardium stained red, and infarcted tissue appeared white. Images (FIG. 5A) were acquired by an MU130 color-complementary metal-oxide-semiconductor (CMOS) camera (AmScope) equipped on an SMZ168 Stereo Zoom microscope (Motic). The infarct area (white), the area at risk (red and white), and the total left ventricle area from each section were measured using ZEN v3.1 (Zeiss). Ratios of infarct area/area at risk (FIG. 5B) and of area at risk/left ventricle (FIG. 5C) were calculated and expressed as percentages.

### Left Ventricle Echocardiogram

To quantify left ventricle function, we performed echocardiograms on mice before and seven days after myocardial I/R injury. Mice were anesthetized with 2% isoflurane i.n. and placed on a heating pad. Chest hair was removed using an electric shaver and animals were fixated on their backs. Echocardiography loops were recorded in B and two-dimensional-targeted M modes in longitudinal and short-axis views on a Vevo 3100 High-Resolution Imaging System equipped with an MX550D transducer (VisualSonics, Toronto, ON, Canada). Mice were fixed on a heated table and heart rate was monitored during the procedure. Systole and diastole were defined based on concomitant electrocardiography (ECG) recordings. The end-systolic time point for left ventricle diameter measurement was defined as the maximum ventricle contraction just before the complete closure of the aortic valve. End-diastole was defined as the maximum left ventricle dilation and filling just before mitral valve closing (when visible) and aortic valve opening. Left ventricular ejection fraction was determined by left ventricle tracing relating the end-systolic left ventricle area as the minimal left ventricle cross-sectional area to the end-diastolic left ventricle area as the maximum left ventricle cross-sectional area in long-axis views. Fractional shortening was assessed by using VevoLab software (VisualSonics).

### Pharmacokinetics

Pharmacokinetics was performed through a service provided by the Shanghai Institute of Materia Medica. Nexinhib20 (160 mM, 5 μL in DMSO mixed with 95 μL 10% Trappsol per mouse) was administered i.p. to three 18-19 g male mice. Blood samples (20 μL) were collected at 3 min, 15 min, 45 min, 2 h, 4 h, 8 h, and 24 h through femoral vein phlebotomy. 200 μL of methanol:acetonitrile (1:1, v/v) with internal standard was added to 20 sL of plasma and vortexed thoroughly. After centrifuging at 11000 rpm, RT for 5 min, 20 μL of the supernatant was collected and mixed with 20 μL of water for analysis. Samples were analyzed by a TQ-S triple quadrupole mass spectrometer (Waters, Milford, MA, USA). An Acquity Uplc Beh C18 Column (1.7 μm, 2.0 mm×50 mm, Waters) was used for the analysis. Gradient elution was applied consisting of 5 mM aluminum ammonium sulfate dodecahydrate containing 0.1% formic acid and methanol:acetonitrile (1/9, v/v) containing 0.1% formic acid.

### Statistics

Statistical analysis was performed using PRISM software (version 8.30, GraphPad Software). Data analysis was performed using student's t-test, one-way ANOVA followed by Tukey's multiple comparisons test, or 2-way ANOVA followed by Šidák's or Tukey's multiple comparisons test, which are indicated in Figure Legends. P values less than 0.05 were considered significant.

### Results

### Nexinhib20 Inhibits IL-8-Induced Neutrophil Adhesion.

Nexinhib20 inhibits exocytosis without inducing apoptosis or cell death. Here, to further analyze whether Nexinhib20 could be toxic to neutrophils, we tested the viability of neutrophils after Nexinhib20 treatment using flow cytometry. We showed that neutrophil viability remained close to 100% even when incubated with 100 μM Nexinhib20 for one hour at room temperature (RT, FIG. 6A). This is consistent with the previous study that Nexinhib20 did not induce a significant increase in cell death after 1 and 4 hours of incubation compared to DMSO vehicle controls. We incubated neutrophils with 10 μM Nexinhib20 for one hour at room temperature in most of our cellular experiments unless stated otherwise.

To assess the impact of Nexinhib20 on neutrophil adhesion, we performed microfluidic assays as described previously. As expected, neutrophils rolled on the substrate of human P-selectin and ICAM-1 under a shear stress of 6 dyn·cm² (FIG. 1A, upper left), which is a typical shear stress in postcapillary venules that commonly show neutrophil recruitment during inflammation. Upon addition of IL-8 to the substrate, neutrophils stopped rolling (arrest), and reduced the 100-second rolling distance from about 200 μm to about 40 μm (FIG. 1A, bottom left). When neutrophils were incubated with Nexinhib20 before the perfusion, they failed to arrest (FIG. 1A, bottom right). After quantifying the rolling velocity of these neutrophils (FIGS. 1B, 1C), we found that IL-8 stimulation did not slow down the rolling velocity of Nexinhib20-treated neutrophils. After 10 minutes of rolling and 5 minutes of washing, we quantified the arrested neutrophils, and found that Nexinhib20 significantly decreased the number of arrested neutrophils from about 200 cells per field-of-view to about 20 cells per field-of-view (about 90%, FIG. 1D). Thus, Nexinhib20 inhibited adhesion of human neutrophils to P-selectin and ICAM-1 in a microfluidic model of physiological flow conditions.

### Nexinhib20 Limits β2 Integrin Exocytosis and Activation.

Since β2 integrins are critical for neutrophil adhesion, Nexinhib20 was tested for its potential to inhibit β2 integrin expression and activation on neutrophils. Nexinhib20 was developed as a neutrophil exocytosis inhibitor that decreases exocytosis of the integrin αMβ2 (Mac-1, CD11b/CD18) a subunit CD11b. It has been shown that after 30 mins of stimulation with granulocyte-macrophage colony-stimulating factor (GM-CSF) and fMLP, CD11b on human neutrophils was upregulated to about 2-fold compared to unstimulated cells. Pretreatment with Nexinhib20 diminished this CD11b upregulation.

Here, we assessed the effect of Nexinhib20 on total β2 subunit (CD18) surface expression (FIG. 2E). We found that total CD18 expression was upregulated by about 40% after 10 min with 1 μg·mL⁻¹ IL-8 stimulation. As expected, this β2 integrin exocytosis was inhibited significantly by Nexinhib20 treatment (FIG. 2E). Secondly, we tested β2 integrin activation, which has two major conformational changes of the β2 integrin extracellular domain—headpiece-opening to acquire high-affinity (H⁺) binding to ligands and extension (E⁺) that allows binding ligands in trans. H⁺ and E⁺ β2 integrins can be monitored by using the conformation-specific antibodies mAb24 and KIM127, respectively. Both lymphocyte function-associated antigen 1 (LFA-1, CD11a/CD18, αLβ2) and Mac-1 were detected. Time-resolved flow cytometry showed that IL-8 induced dramatic increased of both mAb24 (FIG. 2A) and KIM127 (FIG. 2B) binding, and that Nexinhib20 treatment inhibited these effects. Since the time-resolved flow cytometry cannot remove free mAb in the cell suspension which generates background noise, we also use standard flow cytometry with fixation and washing to remove free mAb and get more accurate quantification (FIGS. 2C-2G). Isotype control (mouse IgG1) was used to determine background noise in the standard flow cytometry assay. After 10 min of 1 μg·mL⁻¹ IL-8 stimulation, we found that mAb24 staining increased to about 10-fold, and KIM127 staining increased to about 2-fold. Nexinhib20 inhibited the IL-8-induced elevation of mAb24 (FIG. 2C) by about 75% and KIM127 (FIG. 2D) by about 20%. Since TS1/18 (FIG. 2E) is the same isotype (mouse IgG1) as mAb24 and KIM127 and the same secondary antibody was used, the percentage of high-affinity and extended β2 integrins can be calculated by dividing the MFI of mAb24 and KIM127, respectively, by the MF of TS1/18. We found that Nexinhib20 reduced the percentage of high-affinity (mAb24, FIG. 2F) but not extended β2 integrins (KIM127, FIG. 2G), suggesting that Nexinihib20 inhibits β2 integrin high-affinity activation but not extension activation. These results demonstrated that Nexinhib20 significantly limited both the exocytosis and activation of β2 integrins on human neutrophils, which are critical events for neutrophil adhesion.

### Nexinhib20 Inhibits Chemokine-Induced Calcium (Ca²⁺) Flux.

Intracellular Ca²⁺ transients are involved in the chemokine-triggered integrin inside-out activation signaling pathway. Ca²⁺ and diacylglycerol (DAG) activate Rap1 GTPases, which are critical for integrin inside-out activation, through calcium and DAG-regulated guanine nucleotide exchange factors (CalDAG-GEFs). The short inside-out Ca²⁺ signal can be triggered by IL-8 through its receptor CXCR2. The disassociated Gβγ activates Ras-related C3 botulinum toxin substrate 1 (Rac-1) and phospholipase C β (PLCβ) sequentially and induces intracellular Ca²⁺ flux. Using the intracellular Ca²⁺ dye Fluo-4 and time-resolved flow cytometry, we evaluated transient elevation of Fluo-4 fluorescence in neutrophils upon IL-8 stimulation (FIG. 2H, the red trace). Nexinhib20 treatment potently blocked the IL-8-induced Ca²⁺ signal (FIG. 2H, the blue trace; FIG. 2I).

### Nexinhib20 Suppresses Rac-1 Activation in Cells.

In the integrin inside-out activation signaling pathway, Rac-1 is an upstream signaling molecule of intracellular Ca²⁺ flux. In this pathway, the activation of G-protein-coupled receptors dissociates G protein to Gα and Gβγ subunits. Gβγ activates Rac-1 through P-Rex1 and Vav1, then activates phospholipase C β2 (PLCβ2) and PLCβ3, induces intracellular Ca²⁺ flux and downstream signaling molecules mentioned above to activate 02 integrins. Rac-1 knockout neutrophils showed defects in inside-out signaling-triggered adhesion. Thus, we tested if Nexinhib20 can inhibit Rac-1. The neutrophil-like cell line HL60 and HL60 cells stably expressing CXCR2 (HL60-CXCR2) were used in the Rac-1 activity assays. Using the p21-activated kinase 1-p21 binding domain (PAK-PBD) bead pull-down assay, which enriches for the active GTP form of Rac-1 (Rac-1-GTP), followed by anti-Rac-1 western blots, we found that Nexinhib20 significantly inhibited the IL-8 (FIGS. 3A-3B) or fMLP (FIGS. 3C-3D) induced Rac-1 activation in HL60-CXCR2 or HL60 cells, respectively. Quantification showed that IL-8 (FIG. 3B) and fMLP (FIG. 3D) stimulation increased the amount of Rac-1-GTP by about 80% and about 50%, respectively, and Nexinhib20 treatment eliminated these increases (FIGS. 3B, 3D). To further confirm our findings in human neutrophils, we performed the Rac-1 pull-down assay using Nexinhib20 or vehicle-treated human neutrophils (FIGS. 3E, 3F). Similar to HL60 data, we showed that IL-8 stimulation increases the amount of Rac-1-GTP by about 60%. Nexinhib20 treatment eliminated these increases. Thus, Nexinhib20 inhibited Rac-1 activity in cells, which was consistent with the inhibition of intracellular Ca²⁺ flux and integrin activation in neutrophils.

### Nexinhib20 Antagonizes the Rac-1-GTP Interaction.

Nexinhib20 was discovered by a screen for inhibitors of Ras-related protein Rab27a-synaptotagmin-like 1 (SYTL1 or JFC1) interaction and was expected to directly bind Rab27a by molecular docking analysis. Rac-1 is known to interact with JFC1 as well. Thus, we hypothesized that Nexinhib20 directly binds Rac-1 or competes for Rac-1-GTP binding. To test this, we performed in vitro binding assays using purified His-tagged Rac-1 protein (FIG. 3E). Incubating with the non-hydrolysable GTP analog GTPγS produced active Rac-1-GTP that was enriched by PAK-PBD beads (FIG. 3E, the second column, vehicle control). His-tagged Rac-1 protein incubated with GDP was used as a negative control (FIG. 3E, the first column). In the presence of Nexinhib20, the binding of His-tagged Rac-1 and GTPγS was significantly inhibited in a dose-dependent manner (FIG. 3E). After calculating the fitting curve of the inhibition percentage, we found that the IC50 of Nexinhib20 to Rac-1-GTP binding was about 29.3 μM. These data suggested that Nexinhib20 could antagonize the Rac-1-GTP interaction and may directly bind to Rac-1. This direct inhibition indicated that the upstream P-Rex1 and Vav1 for Rac-1 activation might not be relevant in the Nexinhib20 inhibition of neutrophil integrin activation.

### Nexinhib20 Limits Adhesion-Independent Human Neutrophil ROS Production.

Nexinhib20 was shown to inhibit neutrophil extracellular superoxide anion production by about 50%. Here we tested the effect of Nexinhib20 on neutrophil total ROS production and its adhesion-dependency (FIGS. 6B-6C). After 50 min of 100 nM PMA stimulation, Nexinhib20-treated neutrophils showed significantly reduced total ROS production compared to vehicle controls (FIG. 6B). Interestingly, this inhibition is not adhesion-dependent because CD18 blockade, which reduces adhesion and spreading of neutrophils, did not inhibit ROS production in both vehicle control neutrophils (FIG. 6C) and Nexinhib20-treated neutrophils (data not shown).

### Nexinhib20 Reduces Neutrophil Recruitment to the Coronary Artery During Reperfusion.

Neutrophils are critically involved in myocardial I/R injury. Intravital imaging has shown that neutrophils are recruited abundantly to the coronary artery 60 minutes after reperfusion. To test whether Nexinhib20 inhibits neutrophil recruitment in myocardial I/R injury in vivo, we performed multi-photon imaging on explanted hearts after 35 minutes of ischemia and 60 minutes of reperfusion (FIGS. 4A-4G). To test this possibility, LysM-EGFP mice were used in our study, and were also used to monitor neutrophil recruitment in hearts. We observed profound accumulation of LysM-GFP⁺ cells in the coronary artery in vehicle controls (FIG. 4A, left panel). To mimic a prevention treatment of reperfusion injury before the primary percutaneous coronary intervention, which is feasible in clinics, Nexinhib20 was administered 30 minutes before the reperfusion. Nexinhib20 treatment significantly reduced the number of LysM-GFP⁺ leukocytes (FIG. 4A, right panel). Quantification of EGFP fluorescence in coronary arteries confirmed that Nexinhib20 significantly limited LysM-GFP⁺ leukocyte recruitment to the coronary artery during reperfusion (FIG. 4B).

Although macrophages and monocytes may also be highlighted by EGFP in the LysM-EGFP mice, about 90% of EGFP positive cells in I/R heart are Ly6G⁺ neutrophils. To further explore the components of LysM-GFP⁺ leukocytes in our experimental setting, we used flow cytometry to quantify the percentage of Ly6G⁺ neutrophils in the I/R heart and blood circulation in mice treated with Nexinhib20 or not (FIGS. 4C-4E). Consistent with the multi-photon imaging data (FIGS. 4A-4B), there were fewer LysM-EGFP⁺ leukocytes recruited to the heart and more LysM-EGFP⁺ leukocytes retained in the blood circulation of Nexinhib20-treated mice compared to vehicle controls (FIG. 4C). In heart LysM-EGFP⁺ leukocytes, about 73 to about 95% of them were Ly6G⁺ neutrophils, regardless of Nexinhib20 treatment (FIG. 4D). In blood LysM-EGFP⁺ leukocytes, about 88 to about 98% of them were Ly6G⁺ neutrophils, regardless of Nexinhib20 treatment (FIG. 4E).

If CD45⁺Ly6G⁺ cells are defined as neutrophils, about 6000 neutrophils were recruited to the heart after 35-minute ischemia and 60-minute reperfusion in mice administered vehicle control (FIG. 4F). Nexinhib20 administration significantly reduced heart neutrophil counts to about 2000 (FIG. 4F). The percentage of neutrophils in heart leukocytes was also reduced from about 50% to about 30% by Nexinhib20 administration (FIG. 4F). Since Nexinhib20 limited neutrophil recruitment, neutrophils retained in the blood circulation were doubled in Nexinhib20-treated myocardial I/R mice compared to vehicle controls (FIG. 4G). These data suggest that a compound of Formula 1, in particular Nexinhib20 can inhibit neutrophil recruitment in vivo in this mouse preclinical model of myocardial I/R injury.

### Nexinhib20 Prevents Myocardial I/R Injury.

Neutrophils mediate cardiomyocyte death by causing vascular plugging, releasing degradative enzymes, and generating ROS. Since we showed that Nexinhib20 limits neutrophil recruitment to the coronary artery and Nexinhib20 was discovered as a neutrophil exocytosis inhibitor that inhibits degradative enzyme release and ROS production (FIG. 6B), we reasoned that it might be useful as a dual-functioning drug to treat myocardial I/R injury. As expected, we found that Nexinhib20 administration significantly decreased infarct size (white area in the TTC-phthalo-blue-staining) after myocardial I/R injury compared to no-drug and vehicle controls (FIG. 5A). Sham controls with little to no infarction were shown as well. Quantification of infarct area/area at risk ratios showed that Nexinhib20 significantly reduced the infarct area percentage from about 50%, which were shown in mice administered with vehicle, to about 40% (FIG. 5B). Quantifications of the area of risk percentage confirmed the stability and reproducibility of our surgical procedure (FIG. 5C). These data suggested that Nexinhib20 has potential to treat myocardial I/R injury.

Then we quantified heart function using a left ventricle echocardiogram. We performed the echocardiogram before and 7 days after myocardial I/R injury. Ejection fraction and fractional shortening were measured to quantify left ventricle function (FIGS. 5D and E). Ejection fraction is a measurement, expressed as a percentage, of how much blood the left ventricle pumps out with each contraction. Fractional shortening shows the percentage of size differences of the left ventricle as a parameter of how well the left ventricle is contracting, i.e., reducing its size during systole. We found that in the vehicle-treated mice, the ejection fraction (FIG. 5D) and fractional shortening (FIG. 5E) were reduced by about 25% and about 30%, respectively, after myocardial I/R injury, indicating a loss of left ventricle function. In Nexinhib20-treated mice, there is no significant reduction of either ejection fraction (FIG. 5D) or fractional shortening (FIG. 5E) after myocardial I/R injury. Compared to vehicle-treated mice, Nexinhib20-treated mice have significant improvement in left ventricle function 7 days after myocardial I/R injury (FIGS. 5D and E).

### Definitions

The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising,” “having,” “including,” and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

The term “subject” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject is a human, such as a human being treated or assessed for a disease, disorder or condition, a human at risk for a disease, disorder or condition, a human having a disease, disorder, or condition, and/or human being treated for a disease, disorder, or condition as described herein. In some embodiments, the subject does not suffer from an ongoing autoimmune disease. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age. Values and ranges intermediate to the above recited ranges are also intended to be part of this invention. In addition, ranges of values using a combination of any of the above-recited values as upper and/or lower limits are intended to be included.

“Treatment” as used herein can include treatment of a symptom of an undesirable or aberrant immune response. immune disorder, inflammatory response. inflammation, or cardiovascular event or cardiovascular disease; or treatment of an autoimmune response, disorder or disease, or adverse cardiovascular event or cardiovascular disease. an immune disorder, inflammatory response, inflammation. autoimmune response. Non-limiting examples include rheumatoid arthritis, juvenile rheumatoid arthritis, osteoarthritis, psoriatic arthritis, multiple sclerosis (MS). encephalomyelitis, myasthenia gravis, systemic lupus erythrematosus (SLE), asthma, allergic asthma, autoimmune thyroiditis, atopic dermatitis, eczematous dermatitis. psoriasis. Sjögren's Syndrome, Crohn's disease. aphthous ulcer. iritis, conjunctivitis, keratoconjunctivitis, ulcerative colitis (UC), inflammatory bowel disease (BD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia. idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis. uveitis posterior, interstitial lung fibrosis, Hashimoto's thyroiditis, autoimmune polyglandular syndrome. insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis. antiphospholipid syndrome or an allergy, Behcet's disease. severe combined immunodeficiency (SCID), recombinase activating gene (RAG 1/2) deficiency, adenosine deaminase (ADA) deficiency, interleukin receptor common γ chain (γ_{c}) deficiency, Janus-associated kinase 3 (JAK3) deficiency and reticular dysgenesis; primary T cell immunodeficiency such as DiGeorge syndrome, Nude syndrome. T cell receptor deficiency, MHC class 11 deficiency, TAP-2 deficiency (MHC class I deficiency). ZAP70 tyrosine kinase deficiency and purine nucleotide phosphorylase (PNP) deficiency, antibody deficiencies, X-linked agammaglobulinemia (Bruton's tyrosine kinase deficiency), autosomal recessive agammaglobulinemia, Mu heavy chain deficiency, surrogate light chain (γ5/14.1) deficiency, Hyper-IgM syndrome: X-linked (CD40 ligand deficiency) or non-X-linked. Ig heavy chain gene deletion, IgA deficiency, deficiency of IgG subclasses (with or without IgA deficiency), common variable immunodeficiency (CVID), antibody deficiency with normal immunoglobulins; transient hypogammaglobulinemia of infancy, interferon γ receptor (IFNGR1, IFNGR2) deficiency, interleukin 12 or interleukin 12 receptor deficiency, immunodeficiency with thymoma, Wiskott-Aldrich syndrome (WAS protein deficiency), ataxia telangiectasia (ATM deficiency), X-linked lymphoproliferative syndrome (SH2D1A/SAP deficiency), and hyper IgE syndrome.

A dash (“-”) that is not between two letters or symbols is used to indicate a point of attachment for a substituent. For example, —COOH is attached through the carbon atom.

“Alkyl” as used herein means branched and straight-chain saturated aliphatic hydrocarbon groups having the specified number of carbon atoms. Thus, the term C₁-C₆ alkyl as used herein includes alkyl groups having from 1 to about 6 carbon atoms. When C₀-Cₙ alkyl is used herein in conjunction with another group, for example, phenylC₀-C₄ alkyl, the indicated group, in this case phenyl, is either directly bound by a single covalent bond (C₀), or attached by an alkyl chain having the specified number of carbon atoms, in this case from 1 to 4 carbon atoms. Examples of alkyl groups include, but are not limited to, methyl, ethyl, n-propyl, isopropyl, n-butyl, t-butyl, n-pentyl, and sec-pentyl.

“Aryl” as used herein means an aromatic group containing only carbon in the aromatic ring or rings. Such aromatic groups can be further substituted with carbon or non-carbon atoms or groups. Aryl groups can have 1 to 3 separate, fused, or pendant rings without heteroatoms as ring members. Substitution can include fusion to a 5 to 7-membered saturated cyclic group that optionally contains 1 or 2 heteroatoms independently chosen from N, O, and S, to form, for example, a 3,4-methylenedioxy-phenyl group. Examples of aryl groups include, but are not limited to, phenyl, naphthyl (including 1-naphthyl and 2-naphthyl), and bi-phenyl.

“(Aryl)alkyl” as used herein means a group including an aryl group and an alkyl group as defined above, where the point of attachment of the group is via the alkyl moiety. Examples of (aryl)alkyl group include, but are not limited to, benzyl, phenylethyl, and piperonyl.

“Cycloalkyl” as used herein means a saturated hydrocarbon ring group having the specified number of carbon atoms. Examples of cycloalkyl groups include, but are not limited to, cyclopropyl, cyclobutyl, cyclopentyl, and cyclohexyl as well as bridged or caged saturated ring groups such as norbornane or adamantane.

“Haloalkyl” as used herein means branched and straight-chain saturated aliphatic alkyl group as defined above having the specified number of carbon atoms and substituted with 1 or more halogen atoms, for example up to the maximum allowable number of halogen atoms. Examples of haloalkyl groups include, but are not limited to, trifluoromethyl, difluoromethyl, 2-fluoroethyl, and penta-fluoroethyl.

“Halo” or “halogen” as used herein means fluoro, chloro, bromo, or iodo. A combination of different halogen groups can be present, for example a chlorofluoromethyl group.

“Heteroaryl” as used herein means an aromatic ring group having the specified number of carbon atoms and at least 1, preferably 1 to 4 heteroatoms in the ring, where the heteroatoms can each independently be N, O, S, Si, or P. In an aspect, a heteroaryl group is a stable 5- to 7-membered monocyclic or 7- to 10-membered bicyclic heterocyclic ring group where at least 1 aromatic ring contains from 1 to 4, or from 1 to 3, heteroatoms that can each independently be N, O, or S, with the remaining ring atoms being carbon. When the total number of S and O atoms in the heteroaryl group exceeds 1, these heteroatoms are not adjacent to one another. Preferably, the total number of S and O atoms in the heteroaryl group is 1 or 2. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, and 5,6,7,8-tetrahydroisoquinoline.

“Heteroarylalkyl” as used herein means a group having the indicated number of carbon atoms and including a heteroaryl group and an alkyl group as defined above where the point of attachment of the group is via the alkyl moiety. This term includes, but is not limited to, pyridylmethyl, thiophenylmethyl, and pyrrolyl(1-ethyl).

“Heterocycloalkyl” as used herein means a saturated cyclic ring group having the indicated number of carbon atoms and from 1 to 3 heteroatoms in the ring, wherein the heteroatoms can be N, O, or S. In an aspect, heterocycloalkyl groups have from 3 to 8 ring atoms or 5 to 7 ring atoms and 1, 2, or 3 heteroatoms that can each independently be N, O, or S. Examples of heterocycloalkyl groups include, but are not limited to, morpholinyl, piperazinyl, piperidinyl, pyrrolidinyl, 1,2,4-oxadiazol-3-yl-5(4H)-thione, and 1,2,4-oxadiazol-3-yl-5(4H)-one groups.

“Pharmaceutically acceptable salt” as used herein means a derivative of a compound wherein the parent compound is modified by making an acid or base salt thereof, and further includes pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Pharmaceutically acceptable salts can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable.

The term “substituted” as used herein means that any one or more hydrogens on the designated atom or group is replaced with a selection from the indicated group, provided that the designated atom's normal valence is not exceeded. When a substituent is oxo (i.e., ═O), then 2 hydrogens on the atom are replaced. When aromatic moieties re substituted by an oxo group, the aromatic ring is replaced by the corresponding partially unsaturated ring. For example, a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent. A hydrogen substituent is a hydrogen atom. The number of carbon atoms in a given group does not include any substituents. For example, a 3-cyanophenyl group is a C₆ aryl group.

The groups herein can be optionally substituted with a substituent that is a C₁₋₈ alkyl, C₂₋₈ alkenyl, C₁₋₈ alkoxy, C₁₋₈ alkyl group substituted with 1 to 3 halogen atoms, C₁₋₈ alkoxy substituted with 1 to 3 halogen atoms, a halogen atom, hydroxyl, nitro, cyano, amino, C₁₋₈ alkylamino, C₂₋₈ dialkylamino, or an aralkyl group, as a substituent.

All U.S. and PCT patent publications and U.S. patents mentioned herein are hereby incorporated by reference in their entirety as if each individual patent publication or patent was specifically and individually indicated to be incorporated by reference. In case of conflict, the present application, including any definitions herein, will control.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

We claim:

1. A method for treating or preventing a neutrophil-related acute inflammatory condition in a subject in need thereof, the method comprising:
providing a pharmaceutical composition comprising a compound of Formula 1, and/or a pharmaceutically acceptable salt and/or formulation thereof 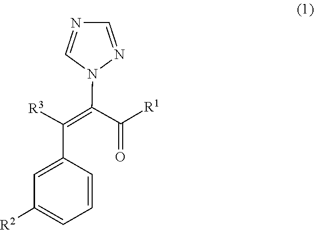
wherein, in Formula 1,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl,
R² is hydrogen, cyano, halo, or nitro, and
R³ is hydrogen, cyano, halo, nitro, C₁-C₄ alkyl, C₁-C₄ haloalkyl, C₃-C₆ cycloalkyl, C₂-C₇ heterocycloalkyl, C₆-C₁₂ aryl, or C₂-C₁₁ heteroaryl; and
administering the pharmaceutical composition to the subject.

2. The method of claim 1, wherein the compound of Formula 1 is of Formula 1a 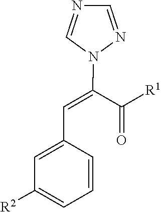
wherein in Formula 1a,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl; and
R² is hydrogen, cyano, halo, or nitro.

3. The method of claim 2, wherein R¹ is C₂-C₆ alkyl; and R² is nitro.

4. The method of claim 3, wherein R¹ is tert-butyl and R² is nitro and the compound is 4,4-dimethyl-1-(3-nitrophenyl)-2-(1H-1,2,4-triazol-1-yl)-1-penten-3-one.

5. A method for treating or preventing myocardial ischemia-reperfusion injury in a subject in need of such treatment, the method comprising:
providing a pharmaceutical composition comprising a compound of Formula 1, and/or a pharmaceutically acceptable salt and/or formulation thereof 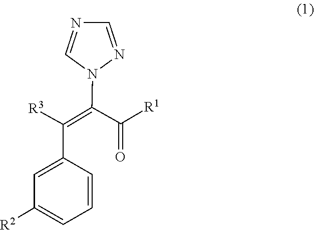
wherein, in Formula 1,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl,
R² is hydrogen, cyano, halo, or nitro, and
R³ is hydrogen, cyano, halo, nitro, C₁-C₄ alkyl, C₁-C₄ haloalkyl, C₃-C₆ cycloalkyl, C₂-C₇ heterocycloalkyl, C₆-C₁₂ aryl, or C₂-C₁₁ heteroaryl; and
administering the pharmaceutical composition to the subject.

6. The method of claim 5, wherein the compound of Formula 1 is of Formula 1a 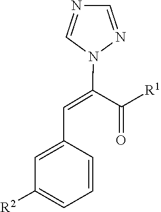
wherein in Formula 1a,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl; and
R² is hydrogen, cyano, halo, or nitro.

7. The method of claim 6, wherein R¹ is C₂-C₆ alkyl; and R² is nitro.

8. The method of claim 7, wherein R¹ is tert-butyl; and R² is nitro, and the compound is 4,4-dimethyl-1-(3-nitrophenyl)-2-(1H-1,2,4-triazol-1-yl)-1-penten-3-one.

9. The method of claim 1, wherein the composition inhibits neutrophil exocytosis and neutrophil adhesion by limiting β2 integrin activation.

10. The method of claim 1, wherein the composition inhibits interleukin 8 (IL-8)-induced β2 integrin-dependent human neutrophil adhesion under flow.

11. The method of claim 1, wherein the composition suppresses intracellular calcium flux and β2 integrin activation after IL-8 stimulation.

12. The method of claim 1, wherein the subject is human.

13. A pharmaceutical composition for treating or preventing a neutrophil-related acute inflammatory condition in a subject in need thereof, comprising a compound of Formula 1, and/or a pharmaceutically acceptable salt and/or a formulation thereof, 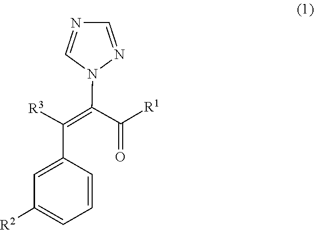
wherein, in Formula 1,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl,
R² is hydrogen, cyano, halo, or nitro, and
R³ is hydrogen, cyano, halo, nitro, C₁-C₄ alkyl, C₁-C₄ haloalkyl, C₃-C₆ cycloalkyl, C₂-C₇ heterocycloalkyl, C₆-C₁₂ aryl, or C₂-C₁₁ heteroaryl; and
administering the pharmaceutical composition to the subject.

14. The composition of claim 13, wherein the compound of Formula 1 is of Formula 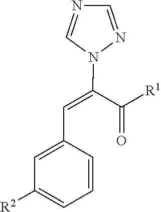
wherein in Formula 1a,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl; and
R² is hydrogen, cyano, halo, or nitro.

15. The composition of claim 14, wherein R¹ is tert-butyl; and R² is nitro, and the compound is 4,4-dimethyl-1-(3-nitrophenyl)-2-(1H-1,2,4-triazol-1-yl)-1-penten-3-one.

16. A pharmaceutical composition for treating or preventing myocardial ischemia-reperfusion injury in a subject in need thereof, the composition comprising a compound of Formula 1, and/or a pharmaceutically acceptable salt and/or a formulation thereof, 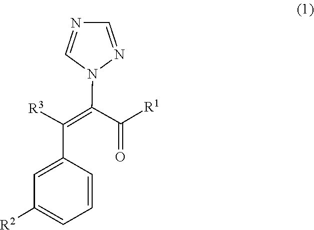
wherein, in Formula 1,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl,
R² is hydrogen, cyano, halo, or nitro, and
R³ is hydrogen, cyano, halo, nitro, C₁-C₄ alkyl, C₁-C₄ haloalkyl, C₃-C₆ cycloalkyl, C₂-C₇ heterocycloalkyl, C₆-C₁₂ aryl, or C₂-C₁₁ heteroaryl; and
administering the pharmaceutical composition to the subject.

17. The composition of claim 16, wherein the compound of Formula 1 is of Formula 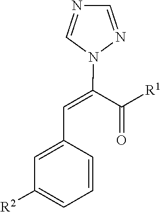
wherein in Formula 1a,
R¹ is hydrogen, cyano, halo, nitro, C₁-C₈ alkyl, C₁-C₈ haloalkyl, C₃-C₆ cycloalkyl, C₂-C₇ heterocycloalkyl, C₆-C₁₂ aryl, or C₂-C₁₁ heteroaryl; and
R² is hydrogen, cyano, halo, or nitro.

18. The composition of claim 17, wherein R¹ is C₂-C₆ alkyl; and R² is nitro.

19. The composition of claim 18, wherein R¹ is tert-butyl; and R² is nitro, and the compound is 4,4-dimethyl-1-(3-nitrophenyl)-2-(1H-1,2,4-triazol-1-yl)-1-penten-3-one.
